(19) United States
(12) Patent Application Publication
(10) Pub. No.: US 2025/0325335 A1
(43) Pub. Date: Oct. 23, 2025

(54) USER INTERFACE FRAMEWORK FOR ANNOTATION OF MEDICAL PROCEDURES

(71) Applicant: Intuitive Surgical Operations, Inc., Sunnyvale, CA (US)

(72) Inventors: Yihan Bao, Santa Clara, CA (US); Akash Aggarwal, San Jose, CA (US); Bikram Basnet, Mountain View, CA (US); Jamie Leong, New York, NY (US); Daniel Pacheco-Maldonado, Atlanta, GA (US)

(73) Assignee: Intuitive Surgical Operations, Inc., Sunnyvale, CA (US)

(21) Appl. No.: 19/181,108

(22) Filed: Apr. 16, 2025

Related U.S. Application Data
(60) Provisional application No. 63/636,040, filed on Apr. 18, 2024.

(51) Int. Cl. A61B 34/00; A61B 34/30; A61B 90/00; G06V 10/764; G06V 10/94; G06V 20/40; G06V 20/50; G16H 30/40; G16H 40/67

(52) U.S. Cl. CPC A61B 34/25; A61B 34/30; A61B 90/361; G06V 10/764; G06V 10/945; G06V 20/41; G06V 20/50; G16H 30/40; G16H 40/67; A61B 2090/373; G06V 2201/03

(57) ABSTRACT

A user interface framework for annotation of medical procedures is provided. A system receives a video stream of a medical procedure performed during a medical session with a robotic medical system and identifies a type of the medical procedure and a phase. The system determines, based on the type of the medical procedure and the phase, a plurality of tasks and display an annotation interface with the plurality of tasks. The system receives, via the annotation interface, a selection of a first type of task and an indication of a start and a stop time and identify frames of the video stream that correspond to the start and stop time for the first type of task. The system constructs, for storage in a data structure, an entry that associates the frames that correspond to the start and stop time with an indication of the first type of task.

### CROSS-REFERENCE TO RELATED APPLICATIONS

This application claims the benefit of priority under 35 U.S.C. § 119 to U.S. Provisional Patent Application No. 63/636,040, filed Apr. 18, 2024, which is hereby incorporated by reference herein in its entirety.

### BACKGROUND

Medical procedures can vary based on their type and the medical tools utilized. With advancements in medical technology, the complexity of procedures increases, making it technically challenging to accurately analyze and track these procedures, thereby making it technically challenging to maintain the performance of such medical procedures.

### SUMMARY

The technical solutions of the present disclosure are directed to an application and a user interface that can serve as a one stop solution for human and machine learning based annotation of medical procedure videos. The technical solutions can allow for, provide, or otherwise facilitate making temporal annotations (e.g., labels, timestamps, descriptions or other metadata) for medical procedure videos, to identify their individual phases and tasks. The technical solutions can utilize an annotation card framework and a user interface to create, edit or validate annotations, allowing multiple case types to be annotated in a single procedure. The technical solutions can provide an annotation card that can list various surgical tasks along with information such as a task description and temporal start and stop parameters associated with the task. The user interface can include or provide a tool bar function for defining boundaries of the procedure's tasks and phases. procedure and tasks and assign annotators to validate the annotations.

At least one aspect of the technical solutions is directed to a system. The system can include one or more processors, coupled with memory. The one or more processors can be configured to receive at least a portion of a video stream of a medical procedure performed during a medical session with a robotic medical system. The one or more processors can be configured to identify, for the at least the portion of the video stream, a type of the medical procedure and a phase of the medical procedure. The one or more processors can be configured to determine, based on the type of the medical procedure and the phase of the medical procedure, a plurality of types of tasks. The one or more processors can be configured to display an annotation interface with the plurality of types of tasks. The one or more processors can be configured to receive, via the annotation interface, a selection of a first type of task of the plurality of types of tasks and an indication of a start time and a stop time for the first type of task. The one or more processors can be configured to identify frames of the at least the portion of the video stream that correspond to the start time and the stop time for the first type of task. The one or more processors can be configured to construct, for storage in a data structure for the medical session, an entry that associates the frames that correspond to the start time and the stop time with an indication of the first type of task.

The one or more processors can be configured to determine a state of the entry based on an expert review protocol and update a field in the entry to indicate the state. The one or more processors can be configured to select an action to validate the entry based on the state and execute the action. The one or more processors can be configured to forward, via a network, the entry to a device for validation, receive, via the network, a validation of the entry according to a review, and store, in the data structure, the entry identified as validated.

The one or more processors can be configured to forward, via a network, the entry to a device for validation, receive, via the annotation interface from the device, a modification to the entry, and update the entry based on the modification. The one or more processors can be configured to identify a plurality of accounts associated with an expert review protocol, and select, based on annotation histories of the plurality of accounts; a first account of the plurality of accounts to validate the entry.

The one or more processors can be configured to identify a plurality of previously validated entries of a plurality of accounts associated with an expert review protocol. The one or more processors can be configured to determine, based on the plurality of previously validated entries and the type of the medical procedure, a first account of the plurality of accounts to validate the entry. The one or more processors can be configured to identify a plurality of video stream files corresponding to the medical session, The one or more processors can be configured to combine the plurality of video stream files to form the at least the portion of the video stream of the medical procedure. The one or more processors can be configured to display the at least portion of the video stream via the annotation interface.

The one or more processors can be configured to identify, using the at least the portion of the video stream, a plurality of phases of the medical procedure comprising the phase. The one or more processors can be configured to identify, for each respective phase of the plurality of phases, a start time of the each respective phase and a stop time of the each respective phase. The one or more processors can be configured to construct, for storage in the data structure, a plurality of entries, each entry of the plurality of entries indicative of the start time of the each respective phase and the stop time of the each respective phase.

The one or more processors can be configured to provide, via the annotation interface, a plurality of modes of the annotation interface. The one or more processors can be configured to display, responsive to a selection from the plurality of modes, a training mode to provide training for annotation of the medical procedure. The one or more processors can be configured to provide, via the annotation interface, a plurality of annotation cards for the plurality of types of tasks of the phase of the medical procedure. The one or more processors can be configured to display, via the annotation interface, responsive to a selection, a first annotation card of the plurality of annotation cards, the first annotation card indicative of the start time and the stop time and comprising a description of the first type of task.

The one or more processors can be configured to identify one or more machine learning (ML) models trained on a plurality of video streams of a plurality of types of medical procedures having a plurality of phases with a plurality of types of tasks. The one or more processors can be configured to identify at least one of the type of the medical procedure or the phase of the medical procedure using the at least the portion of the video stream input into the one or more machine learning (ML) models.

The one or more processors can be configured to identify one or more machine learning (ML) models trained on a plurality of video streams of a plurality of types of medical procedures having a plurality of phases with a plurality of types of tasks identified by a plurality of start times and stop times. The one or more processors can be configured to identify the first type of task and the indication of the start time and the stop time for the first type of task using the one or more machine learning (ML) models. The one or more processors can be configured to identify one or more machine learning (ML) models trained on a plurality of video streams of a plurality of types of medical procedures having a plurality of phases with a plurality of types of tasks. The one or more processors can be configured to determine, using the at least the portion of the video stream input into the one or more machine learning (ML) models, a metric indicative of performance associated with a surgeon performing the medical procedure. The one or more processors can be configured to display the metric via the annotation interface.

At least one aspect of the technical solutions is directed to a system. The method can include identifying, by the one or more processors, for at least a portion of a video stream of a medical procedure performed during a medical session with a robotic medical system, a type of the medical procedure and a phase of the medical procedure. The method can include determining, by the one or more processors, based on the type of the medical procedure and the phase of the medical procedure, a plurality of types of tasks. The method can include receiving, by the one or more processors, via an annotation interface displaying the plurality of types of tasks, a selection of a first type of task of the plurality of types of tasks and an indication of a start time and a stop time for the first type of task. The method can include identifying, by the one or more processors, frames of the at least the portion of the video stream that correspond to the start time and the stop time for the first type of task. The method can include storing, in a data structure for the medical session, an entry that associates the frames that correspond to the start time and the stop time with an indication of the first type of task.

The method can include determining, by the one or more processors, a state of the entry based on an expert review protocol. The method can include updating, by the one or more processors, a field in the entry to indicate the state. The method can include selecting, by the one or more processors, an action to validate the entry based on the state. The method can include executing, by the one or more processors, the action.

The method can include forwarding, by the one or more processors via a network, the entry to a device for validation. The method can include receiving, by the one or more processors via the network, a validation of the entry according to a review. The method can include storing, by the one or more processors in the data structure, the entry identified as validated. The method can include forwarding, by the one or more processors via a network, the entry to a device for validation. The method can include receiving, by the one or more processors via the annotation interface from the device, a modification to the entry. The method can include updating, by the one or more processors, the entry based on the modification.

The method can include identifying, by the one or more processors, a plurality of accounts associated with an expert review protocol. The method can include selecting, by the one or more processors based on annotation histories of the plurality of accounts; a first account of the plurality of accounts to validate the entry. The method can include identifying, by the one or more processors, a plurality of previously validated entries of a plurality of accounts associated with an expert review protocol. The method can include determining, by the one or more processors based on the plurality of previously validated entries and the type of the medical procedure, a first account of the plurality of accounts to validate the entry.

The method can include identifying, by the one or more processors, a plurality of video stream files corresponding to the medical session. The method can include combining, by the one or more processors, the plurality of video stream files to form the at least the portion of the video stream of the medical procedure. The method can include displaying, by the one or more processors, the at least portion of the video stream via the annotation interface. The method can include identifying, by the one or more processors, using the at least the portion of the video stream, a plurality of phases of the medical procedure comprising the phase. The method can include identifying, by the one or more processors, for each respective phase of the plurality of phases, a start time of the each respective phase and a stop time of the each respective phase. The method can include constructing, by the one or more processors, for storage in the data structure, a plurality of entries, each entry of the plurality of entries indicative of the start time of the each respective phase and the stop time of the each respective phase.

The method can include providing, by the one or more processors via the annotation interface, a plurality of modes of the annotation interface. The method can include displaying, by the one or more processors, responsive to a selection from the plurality of modes, a training mode to provide training for annotation of the medical procedure. The method can include providing, by the one or more processors via the annotation interface, a plurality of annotation cards for the plurality of types of tasks of the phase of the medical procedure. The method can include displaying, by the one or more processors via the annotation interface responsive to a selection, a first annotation card of the plurality of annotation cards, the first annotation card indicative of the start time and the stop time and comprising a description of the first type of task.

The method can include identifying, by the one or more processors, one or more machine learning (ML) models trained on a plurality of video streams of a plurality of types of medical procedures having a plurality of phases with a plurality of types of tasks. The method can include identifying, by the one or more processors, at least one of the type of the medical procedure or the phase of the medical procedure using the at least the portion of the video stream input into the one or more machine learning (ML) models.

The method can include identifying, by the one or more processors, one or more machine learning (ML) models trained on a plurality of video streams of a plurality of types of medical procedures having a plurality of phases with a plurality of types of tasks identified by a plurality of start times and stop times. The method can include identifying, by the one or more processors, the first type of task and the indication of the start time and the stop time for the first type of task using the one or more machine learning (ML) models.

The method can include identifying, by the one or more processors, one or more machine learning (ML) models trained on a plurality of video streams of a plurality of types of medical procedures having a plurality of phases with a plurality of types of tasks. The method can include determining, by the one or more processors, using the at least the portion of the video stream input into the one or more machine learning (ML) models, a metric indicative of performance associated with a surgeon performing the medical procedure. The method can include displaying, by the one or more processors, the metric via the annotation interface.

At least one aspect of the technical solutions is directed to a non-transitory computer-readable medium storing processor executable instructions. The processor executable instructions can be such that, when executed by one or more processors, cause the one or more processors to receive at least a portion of a video stream of a medical procedure performed during a medical session with a robotic medical system. The processor executable instructions can be such that, when executed by one or more processors, cause the one or more processors to identify, for the at least the portion of the video stream, a type of the medical procedure and a phase of the medical procedure. The processor executable instructions can be such that, when executed by one or more processors, cause the one or more processors to determine, based on the type of the medical procedure and the phase of the medical procedure, a plurality of types of tasks. The processor executable instructions can be such that, when executed by one or more processors, cause the one or more processors to display an annotation interface with the plurality of types of tasks. The processor executable instructions can be such that, when executed by one or more processors, cause the one or more processors to receive, via the annotation interface, a selection of a first type of task of the plurality of types of tasks and an indication of a start time and a stop time for the first type of task. The processor executable instructions can be such that, when executed by one or more processors, cause the one or more processors to identify frames of the at least the portion of the video stream that correspond to the start time and the stop time for the first type of task. The processor executable instructions can be such that, when executed by one or more processors, cause the one or more processors to construct, for storage in a data structure for the medical session, an entry that associates the frames that correspond to the start time and the stop time with an indication of the first type of task.

These and other aspects and implementations are discussed in detail below. The foregoing information and the following detailed description include illustrative examples of various aspects and implementations and provide an overview or framework for understanding the nature and character of the claimed aspects and implementations. The drawings provide illustration and a further understanding of the various aspects and implementations and are incorporated in and constitute a part of this specification. The foregoing information and the following detailed description and drawings include illustrative examples and should not be considered as limiting.

### BRIEF DESCRIPTION OF THE DRAWINGS

The accompanying drawings are not intended to be drawn to scale. Like reference numbers and designations in the various drawings indicate like elements. For purposes of clarity, not every component can be labeled in every drawing. In the drawings:

FIG. 1 depicts an example system for annotation of medical procedures using a user interface and application framework.

FIG. 2 illustrates an example of annotation cards displayed in an annotation interface.

FIGS. 3-10 illustrates different example of an annotation interface along with their features and operation modes.

FIG. 11 illustrates an example of a surgical system, in accordance with some aspects of the technical solutions.

FIG. 12 illustrates an example block diagram of an example computer system is shown, in accordance with some aspects of the technical solutions.

FIG. 13 illustrates an example flow diagram of a method for annotation of medical procedures using a user interface and application framework.

FIGS. 14-16 illustrate examples of annotation interfaces along with their features for user interaction.

### DETAILED DESCRIPTION

Following below are more detailed descriptions of various concepts related to, and implementations of, systems, methods, apparatuses for providing a user interface and an application for labeling and annotation of medical procedures. The various concepts introduced above and discussed in greater detail below can be implemented in any of numerous ways.

When seeking to annotate medical procedures, it can be challenging to access different video streams for various procedure segments, seek and provide validations for various annotated procedures or integrate different machine learning tools, as each of these tasks are typically performed using different and often dissimilar tools and applications. Using such applications to complete these tasks can trigger compatibility issues, as converting various file formats can involve specialized tools to improve interoperability among dissimilar applications. In addition, data management can become difficult as video streams and associated annotation data can benefit from more efficient storage solutions, while repeated uploading and downloading of video streams of different procedure phases and tasks can be compute and energy intensive.

The technical solutions of this disclosure overcome these challenges by providing an integrated annotation framework with an application and a user interface that facilitate a more efficient, streamlined and less compute intensive temporal annotation of medical procedures. The technical solutions provide annotation cards to facilitate user-based and machine learning-based annotation of individual phases and tasks of the medical procedures. This framework provides the functionalities for creation, editing, and validation of annotations, allowing for multiple case types and video streams to be annotated within a single procedure and validated by any number of users. Additionally, the interface can include includes toolbar functions for defining task and phase boundaries, improving efficiency and usability.

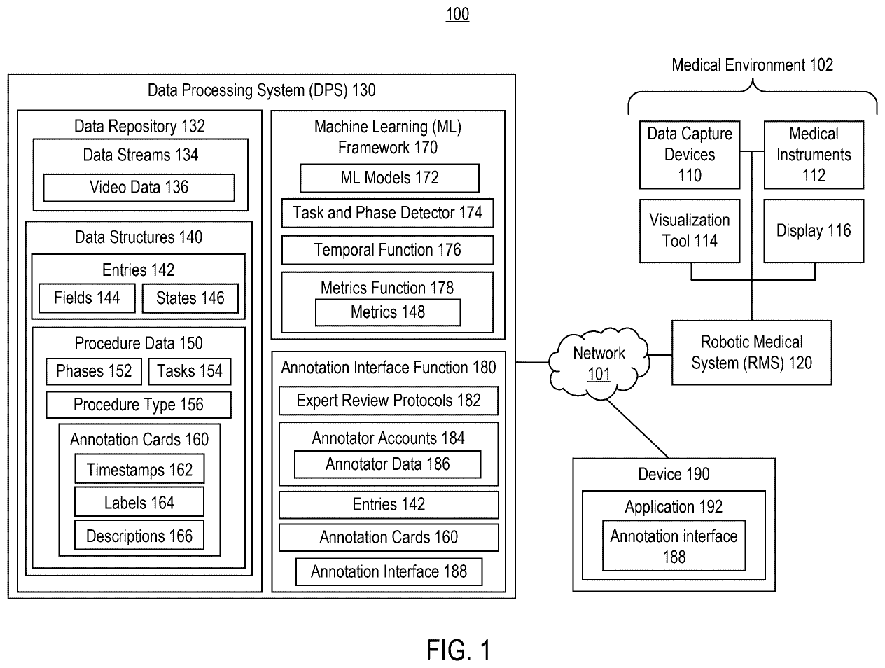

FIG. 1 depicts an example system 100 for annotation of medical procedures using a user interface and application framework. Example system can include a surgical robotic system for performing tasks using medical instruments, such as a robotic medical system 120 used by a surgeon to perform a surgery on a patient. Robotic medical system 120, also referred to as an RMS 120, can be deployed in a medical environment 102. Medical environment 102 can include any space or facility for performing medical procedures, such as a surgical facility, or an operating room. Medical environment 102 can include medical instruments 112 that the RMS 120 can use for performing surgical patient procedures, whether invasive, non-invasive, in-patient, or out-patient procedures.

The medical environment 102 can include one or more data capture devices 110 (e.g., optical devices, such as cameras or sensors or other types of sensors or detectors) for capturing data streams 134, that can include video data 136 of images or a video stream of a surgery. The medical environment 102 can include one or more visualization tools 114 to gather the captured data streams 134 and process it for display to the user (e.g., a surgeon or other medical professional) at one or more displays 116. A display 116 can present data stream 134 (e.g., video data 136 or events or kinematics data of an RMS 120) of an ongoing medical procedure (e.g., an ongoing surgery) performed using the robotic medical system 120 handling, manipulating, holding or otherwise utilizing medical instruments or tools 112 to perform surgical tasks at the surgical site. Coupled with the RMS 120, via a network 101, can be a data processing system (DPS) 130. DPS 130 and a device 190.

DPS 130 can include one or more data repositories 132 storing data streams 134 that can include various video data 136 and other data (e.g., events data, kinematics data or sensor data) as well as one or more data structures 140. Data structures 140 can include entries 142 and procedure data 150. Entries 142 can include fields 144 and states 146 of entries. Procedure data 150 can include data on phases 152 and tasks 154 of medical procedures and medical procedure types 156. Procedure data 150 can include annotation cards 160 that can include one or more timestamps 162, labels 164 or descriptions 166. DPS 30 can include one or more machine learning (ML) frameworks 170 that can include one or more ML models 172, task and phase detectors 174, temporal functions 176 and metrics functions 178 for generating performance metrics 148. DPS 130 can include one or more annotation interface functions (AIF) 180 having or utilizing one or more expert review protocols 182, annotator accounts 184 with annotator data 186, entries 142, annotation cards 160 and annotation interfaces 188. Across the network 101, a device 190 (e.g., a network device of a user or annotator) can include or execute one or more applications 192 utilizing or accessing annotation interface 188 and its features in order to implement, user or validate annotation of medical procedures using the features of the system 100.

Data repository 132 can include various data streams 134 generated by the robotic medical system (RMS) 120, including video data 136, having any type and form of video frames. Data streams 134 can include also any kinematics data, sensor data, or events data from the RMS 120, data capture devices 110, medical instruments 112, visualization tools 114 or displays 116. Annotation interface 188 can be accessed and used via various user devices 190 to access data structures 140, including entries 142 and procedure data 150 at the data repository 132. Using the annotation interface 188, user can access, select, enter, implement or provide various entries 142, including fields 144 and states 146 corresponding to, or including, any procedure data 150 (e.g., phases 152, tasks 154 or any other portion of annotation cards 160.

AIF 180 and ML framework 170 (e.g., ML models 172, along with functions 174, 176 and 178) can use data streams 134, such as inputs into one or more ML models 172, to detect and identify tasks 154 and phases 152, timestamps 162 or temporal points at which tasks 154 or phases 152 begin or end, or any metrics 148 for performance of surgeons with respect to particular tasks 154, phases 152 or medical procedures (e.g., procedure type 156). Machine learning (ML) framework 170 can include any combination of hardware and software for providing a system that integrates ML-based anatomy and instrument models alongside attention mechanisms and rule-based modeling to detect and recognize interactions between medical instruments 112 detected by the ML models 172. ML framework 170 can include one or more ML modules and functions (e.g., 174-178) for implementing various tasks associated with annotation of medical procedures. ML framework 170 can include one or more ML training functions for training ML models 172 using various data, such as data streams 134, procedure data 150, entries 142, metrics 148 or other information or data.

ML framework 170 can be designed and trained to perform various functionalities used for annotation of medical procedures. ML models 142 can be trained or configured to determine task 154 and phase 152, identify timestamps 162 (e.g., start and end times) for tasks 154 and phases 152, apply labels 164 to ML-determined or user-identified phases 152 and tasks 154, or determine performance metrics 148 of surgeons or other medical professionals performing the medial procedure. ML models 172 can utilize various machine learning architectures or mechanisms, such as attention mechanism, which can be implemented using neural networks. ML model 172 attention mechanism can facilitate extraction of spatial and temporal features from the input data streams 134. Attention mechanisms can facilitate or improve the ability or capacity of the ML models 172 to discern, detect or recognize specific phases 152 or tasks 154, identify timestamps 162 for the timing of the start and end points of various phases 152 or tasks 154, apply labels 164 to such identified phases 152 or tasks 154 along with any metrics 148 that can be determined with respect to phases 152, tasks 154 or medical procedure types 156 (e.g., an instance of a medical procedure as a whole).

ML framework 170 can include and provide rule-based modeling to determine and quantify the consistency of motion of features (e.g., medical instruments 112, patient anatomies or other object) in the video data 136. ML framework 170 can include a use image encoders for extracting image features and temporal functions 176 for identifying timing or timestamps 162 of various points in the video data 136 (e.g., the data stream). For example, the ML framework 170 can may utilize attention mechanisms to focus on relevant regions of interest within the video data 136, while also using rule-based modeling to assess the coherence or correlation of detected motion or movements, thus facilitating improved accuracy of the determinations.

Data repository 132 can include one or more data streams 134, such as video data 136 that can include any type of a stream of video frames. Data streams 134 can include any number of video frames such as endoscopic images or data, medical environment video surveillance data, infrared data, ultrasound data or any other data. Data stream 134 can include non-video data including sensor measurements, such as force, torque or biometric data, haptic feedback data, pressure or temperature data, vibration, tension or compression data or command data streams. Data repository 132 can include event data, such as installation data, including data on installation, uninstallation, activation, deactivation, calibration or use of particular medical instruments 112 or other components.

The system 100 can include one or more data capture devices 110 (e.g., video cameras, sensors or detectors) for collecting any data stream 134, that can be used by the users accessing annotation interface 188 or for machine learning and detection of objects, such as medical instruments 112 or detection of phases 152 and tasks 154. Data capture devices 110 can include cameras or other image capture devices for capturing video data 136 (e.g., videos or images) from a particular viewpoint within the medical environment 102. The data capture devices 110 can be positioned, mounted, or otherwise located to capture content from any viewpoint that facilitates the data processing system 130 capturing various surgical tasks or actions.

Data capture devices 110 can include any sensors, still or motion video imaging devices, infrared imaging devices, visible light imaging devices, intensity imaging devices (e.g., black, color, grayscale imaging devices, etc.), depth imaging devices (e.g., stereoscopic imaging devices, time-of-flight imaging devices, etc.), medical imaging devices such as endoscopic imaging devices, ultrasound imaging devices, etc., non-visible light imaging devices, any combination or sub-combination of the above mentioned imaging devices, or any other type of imaging devices that can be suitable for the purposes described herein. Data capture devices 110 can include cameras that a surgeon can use to perform a surgery and observe manipulation components within a purview of field of view suitable for the given task performance.

Data capture devices 110 can capture, detect, or acquire sensor data, such as videos or images, including for example, image frames, still images, vector images, bitmap images, other types of images, or combinations thereof. Data capture devices 110 can capture the images at any suitable predetermined capture rate or frequency. Settings, such as zoom settings or resolution, of each of the data capture devices 110 can vary as desired to capture suitable images from any viewpoint. For instance, data capture devices 110 can have fixed viewpoints, locations, positions, or orientations. The data capture devices 110 can be portable, or otherwise configured to change orientation or telescope in various directions. The data capture devices 110 can be part of a multi-sensor architecture including multiple sensors, with each sensor being configured to detect, measure, or otherwise capture a particular parameter (e.g., sound, images, or pressure).

Display 116 can show, illustrate or play data streams 134, including video data 136, in which medical tools 112 at or near surgical sites are shown. For example, display 116 can display a rectangular image (e.g., a frame of a video data 136) of a surgical site along with at least a portion of medical instruments 112 being used to perform surgical tasks. Display 116 can provide compiled or composite images generated by the visualization tool 114 from a plurality of data capture devices 110 to provide visual feedback from one or more points of view.

The visualization tool 114 that can be configured or designed to receive any number of different data streams 134 from any number of data capture devices 110 and combine them into a single data stream displayed on a display 116. The visualization tool 114 can be configured to receive a plurality of data stream parts and combine the plurality of data stream parts into a single data stream 134 for display on a display 116 or a display of a user device 190. For instance, the visualization tool 114 can receive a visual sensor data from one or more medical tools 112, sensors or cameras with respect to a surgical site or an area in which a surgery is performed. The visualization tool 114 can incorporate, combine or utilize multiple types of data (e.g., positioning data of a medical tool 112 along sensor readings of pressure, temperature, vibration or any other data) to generate an output to present on a display 116. Visualization tool 114 can present locations of medical tools 112 along with locations of any reference points or surgical sites, including locations of anatomical parts of the patient (e.g., organs, glands or bones).

Medical instruments or tools 112 can be any type and form of tool or instrument used for surgery, medical procedures or a tool in an operating room or environment. Medical tool 112 can be imaged by, associated with or include an image capture device. For instance, a medical tool 112 can be a tool (e.g., a scalpel) for making incisions, a tool (e.g., a needle and a thread) for suturing a wound, an endoscope for visualizing organs or tissues, an imaging device, forceps, scissors, retractors, graspers, or any other tool or instrument to be used during a medical procedure. Medical instruments or tools 112 can include hemostats, trocars, surgical drills, suction devices or any instruments for use during a surgery. The medical tool 112 can include other or additional types of therapeutic or diagnostic medical imaging implements. The medical tool 112 can be configured to be installed in, coupled with, or manipulated by an RMS 120, such as by manipulator arms or other components for holding, using and manipulating the medical instruments or tools 112.

RMS 120 can be a computer-assisted system configured to perform a surgical or medical procedure or activity on a patient via or using or with the assistance of one or more robotic components or medical tools 112. RMS 120 can include any number of manipulator arms for grasping, holding or manipulating various medical tools 112 and performing computer-assisted medical tasks using medical tools 112 controlled by the manipulator arms.

Video data 136, including any images or videos captured by a medical tool 112 (e.g., endoscopic camera) can be sent to the visualization tool 114. The robotic medical system 120 can include one or more input ports to receive direct or indirect connection of one or more auxiliary devices. For example, the visualization tool 114 can be connected to the RMS 120 to receive the images from the medical instrument 112 when the medical instrument 112 is installed in the RMS 120 (e.g., on a manipulator arm of the RMS 120 that is used for moving, managing or otherwise handing medical instruments 112). The visualization tool 114 can combine the data streams 134 from the data capture devices 110 and the medical tool 112 into a single combined data stream 134 for use by the ML framework 170.

The system 100 can include a data processing system 130. The data processing system 130 can be deployed in or associated with the medical environment 102, or it can be provided by a remote server or be cloud-based. The data processing system 130 can include an annotation interface 188 designed, constructed and operational to communicate with one or more component of system 100 via network 101, including, for example, the robotic medical system 120. Data processing system 130 can be implemented using instructions stored in memory locations and processed by one or more processors, controllers or integrated circuitry. Data processing system 130 can include functionalities, computer codes or programs for executing or implementing any functionality of ML framework 170, including any ML models 172 along with any associated functions or features for user interface operation and annotation of medical procedures.

ML model 172 can include any combination of hardware and software for performing any tasks that can be used in annotation of medical procedures. ML model 172 can include the training, configuration or functionality to detect and identify tasks 154 and phases 152 within a medical procedure, identify timestamps 162 to label the starting and ending temporal points of such tasks 154 or phases 152 and determine any performance metrics 148 for the surgeon performing the medical procedure. ML model 172 can include a neural network model that can utilize an image encoder to detect features of a video data 136. ML model 172 can utilize a task and phase detector 174 function to detect tasks 154 and phases 152. ML model 172 can utilize temporal function 176 to determine timestamps 162 corresponding to temporal starting and ending points of any task 154 or phase 152, including the starting and ending point of a medical procedure. ML model 172 can utilize a metrics function 178 to determine metrics 148 for a surgeon or other doctors performing various tasks 154, phases 152 or procedure types 156. ML model 172 can be trained or configured to generate and provide a confidence score or a confidence level of determinations, including the confidence level in the determined metric 148 for the performance, or a confidence score or a level corresponding to a determination of a particular phase 152, task 154 or medical procedure type 156.

ML model 172 can include or utilize any ML-based architecture. For instance, ML model 172 can include and utilize transformers or transformer-based architectures, such as a spatial-temporal transformers or a graphical neural network with transformers to detect, recognize, or generate objects or features. ML model 172 can detect or recognize medical tools 112, tasks 154 or phases 152. ML model 172 can generate or apply timestamps 162 for marking various phases or tasks, apply labels 164 to mark such phases, stamps or medical tools 112, or generate descriptions 166, such as texts describing tasks 154, phases 152 or events. ML model 172 can be trained to provide real-time annotation, such that data stream 134 of a video is being annotated by the ML model 172 in real-time during the procedure.

ML model 172 can include any combination of hardware and software, including machine learning features and architectures performing tasks related to annotation of medical procedures (e.g., 156). ML models 172 can be trained or configured to assist users associated with annotator accounts 184 to access annotation interface 188 and enter, generate or update various data, such as entries 142 or any procedure data 150. ML model 172 can utilize an image encoder or spatial-temporal transformer to detect or identify tasks 154 or phases 152 from video data 136. ML model 172 can utilize task and phase detector 174 to determine or detect types of medical procedures, types of phases 152 and types of tasks 154, as well as apply labels 164 to such identified sections of the medical procedure (e.g., 156). ML model 172 can be trained to detect and mark using timestamps 162 any specific portion (e.g., task 154 or phase 152) of a medical procedure, including the start and end of any such portion. ML model 172 make such determinations using video data 136 or events data (e.g., installation timing of a medical instrument 112) as well as kinematics data (e.g., movement data on a medical tool 112). ML model 172 can be trained or configured to determine metrics 148 based on trained detection of tasks 154 and phases 152 and comparison of such tasks and phases with those from the video recording, to assess or determine the performance (e.g., score or quality) of the phase 152 or task 154 performed by a surgeon.

ML model 172 can include support vector machines (SVMs) that can facilitate predictions (e.g., anatomical, instrument, object, action or any other) in relation to class boundaries, random forests for classification and regression tasks, decision trees for prediction trees with respect to distinct decision points, K-nearest neighbors (KNNs) that can use similarity measures for predictions based on characteristics of neighboring data points, Naïve Bayes functions for probabilistic classifications, logistic or linear regressions, or gradient boosting models. ML model 172 can include neural networks, such as deep neural networks configured for hierarchical representations of features, convolutional neural networks (CNNs) for image-based classifications and predictions, as well as spatial relations and hierarchies, recurrent neural networks (RNNs) and long short-term memory (LSTM) networks for determining structures and processes unfolding over time. ML model 172 can include or utilize transformers or transformer-based architectures, such as a spatial-temporal transformers or a graphical neural network with transformers to make determinations or perform any actions.

The ML model 172 can be trained by an ML model trainer which can include any combination of hardware and software for training ML models 172. Machine learning (ML) trainer can include or generate ML models 172, each of which can be trained using large training datasets that can include any number of various data streams 134, annotation cards 160, procedure data 150 or entries 142. ML model trainer can utilize inputs from users (e.g., annotators) to improve the performance of the ML model 172 by retraining the ML model 172 using the user (e.g., annotator) updated data. ML model 172 can include the functionality to train any features of the ML models 172 including any combination of data structure 140 entries 142, procedure data 150 or data streams 134 of videos of various medical procedures.

Task and phase detectors 174 can include any combination of hardware and software for detecting tasks and phases. Task and phase detectors 174 can detect tasks 154 and phases 152 based on user selections or machine learning. Task and phase detectors 174 can use one or more ML models 172 to detect phases 152, tasks 154 or medical procedures, along with their respective types, starting and ending points. Taks and phase detectors 174 can include the functionality that can be utilized by users of annotation interface 188 on device 190 to identify and mark the tasks 154 and phases 152.

Temporal functions 176 can include any combination of hardware and software for determining temporal points (e.g., timestamps 162) at particular points of the video data 136 (e.g., video stream of a medical procedure or a medical procedure type 156). Temporal functions 176 can identify temporal points at which task 154 or phase 152 starts or ends, at which a description 166 starts or ends or when a label 164 appears. Temporal functions 176 can be implemented using ML models 172 or can be entered by users of annotation interface 188 using devices 190.

Metrics function 178 can include any combination of hardware and software for determining performance metrics 148 of any task 154, phase 152 or a medical procedure. Metrics function 178 can generate metrics 148, quantifying or indicating the level of performance of a surgeon or a medical professional performing a medical procedure. Determined metric 148 can correspond to a medical procedure as a whole, to one or more medical tasks 154 or one or more medical phases 152. Metrics function 178 can be updated or operated using annotation interface 188 (e.g., based on user selection or entry) or based on determinations from a ML model 172.

The data repository 132 can include one or more data files, data structures, arrays, values, or other information that facilitates operation of the data processing system 130. Data stream 134 can store entries 142 (e.g., fields 144 or states 146) corresponding to any phase 152, task 154 or any portion of an annotation card 160 (e.g., timestamps 162, labels 164 or descriptions 166). The data repository 132 can include one or more local or distributed databases and can include a database management system. The data repository 132 can include, maintain, or manage a data stream 134. The data stream 134 can include or be formed from one or more of a video stream, image stream, stream of sensor measurements, event stream, or kinematics stream. The data stream 134 can include data collected by one or more data capture devices 110, such as a set of 3D sensors from a variety of angles or vantage points with respect to the procedure activity (e.g., point or area of surgery).

Data stream 134 can include video data 136, which can include a series of video frames formed or organized into video fragments, such as video fragments of about 1, 2, 3, 4, 5, 10 or 15 seconds of a video. Each second of the video can include, for example, 30, 45, 60, 90 or 120 video frames 308, per second. Data stream 134 can include a stream of events data which can include a stream of event data or information, such as packets, which identify or convey a state of the robotic medical system 120 or an event that occurred in association with the robotic medical system 120. Events data can include information on a state of the RMS 120 indicating whether a medical instrument 112 is calibrated, adjusted or includes a manipulator arm installed on an RMS 120. Event data can include data on whether an RMS 120 is fully functional (e.g., without errors) during the procedure. For example, when a medical instrument 112 is installed on a manipulator arm of the RMS 120, a signal or data packet(s) can be generated indicating that the medical instrument 112 has been installed on the manipulator arm of the RMS 120.

Data stream 134 can include a stream of kinematics data, which can include data associated with motion of one or more of the manipulator arms or medical tools 112 (e.g., instruments) attached to the manipulator arms, such as arm movements, locations or positioning. Data corresponding to medical tools 112 can be captured or detected by one or more displacement transducers, orientational sensors, positional sensors, or other types of sensors and devices to measure parameters or generate kinematics information. The kinematics data can include sensor data along with time stamps and an indication of the medical tool 112 or type of medical tool 112 associated with the data stream 134.

DPS 130 can include an annotation interface 188 designed, constructed and operational to communicate with one or more component of system 100 via network 101, including, for example, the RMS 120 or another device 190, such as a client's personal computer. The annotation interface 188 can include or utilize a network interface to establish sessions or connections with devices 190 across a network 101. The annotation interface 188 can include or provide a user interface, such as a graphical user interface, a web browser, a webpage or a web site, a video editing software or a function, or any other interface. The graphical user interface can include, for example, a window for displaying video data 136, or annotation cards 160 that can be displayed individually or overlaid or displayed instead of, alongside or with, or on top of the video data 136. Annotation interface 188 can provide data for presentation via a display, such as a display 116 (e.g., on a device 190, RMS 120 or any other system or device), and can depict, illustrate, render, present, or otherwise provide annotation cards 160 indicating timestamps 162, labels 164, descriptions 166 or any other markings corresponding to entries 142 for phases 152, tasks 154 or any other portion of medical procedures (e.g., 156).

Annotation interface 188 can be used to display or provide access to one or more annotation cards 160. Annotation card 160 can include any combination of hardware and software for providing data on a particular phase 152, task 154 or medical procedure types 156. Annotation card 160 can include a format or a data structure (e.g., data structure 140) that can include or organize various information or data on particular portion of a medical procedure and its annotations (e.g., labels 164, timestamps 162, descriptions 166 or any other annotations, such as a highlights, markings, comments or metadata tags). Annotation card 160 can include any combination of procedure data 150 or entries 142 for any particular annotation (e.g., label 164, description 166 or timestamp 162). Annotation interface 188 can generate and provide access to the annotation card 160 using various options, selections or modes on the annotation interface 188. Annotation cards 160 can include logs of temporal-based surgical annotations specific to individual cases, including temporal-based annotations of individual tasks 154, phases 152 or procedures. Objective performance indicators, such as metrics 148 of performance of a surgeon, can be determined based on the entries in the annotation cards 160.

Annotation cards 160 can include various entries 142 corresponding to information about surgical tasks, task descriptions 166, start and stop parameters (e.g., timestamps 162 marking start and end of tasks 154) and information identifying procedures that the tasks 154 or phases 152 are associated with. Annotation cards 160 can include information about assignment of the annotation responsibilities to various users (e.g., annotators) based on expert review protocol 182. For example, assignment of annotation duties can be done based on availability of annotators or their workload. AIF 180 can determine, based on a user having a workload that is lower than other users, to assign the annotation duty to the given user. Annotation card 160 can also include assignments of annotation duties based on expertise of the user, including for example, the annotator data 186 showing a history of the user working on particular types of tasks 154, phases 152 or medical procedure types 156. Annotation cards 160 can be displayed alongside video stream display to allow the annotators to review the video while performing annotations. Annotation card 160 can include a confidence level in the annotation that the user may provide during the annotation.

Entries 142 can include any type and form of entries, data or information for annotating a medical procedure (e.g., 156). Entries 142 can include objects for entering data or information to annotate or mark one or more video streams or video files according to a medical task 154, phase 152 of a medical procedure or the medical procedure type 156 as a whole. Entry 142 can include, for example, a timestamp 162 for a start or an end of a particular phase 152 or task 154. Entry 142 can include, for example, a state 146 of an entry 142 that can be filled or empty, determined or undetermined, verified, unverified, or awaiting verification. Entries 142 can include fields 144 in which entries 142, such as values or characters indicative of timestamps 162, labels 164 and descriptions 166.

Procedure data 150 can include various information, indications or medical procedure data 150, such as a procedure type 156 or any information or data on phases 152 of the procedure being performed or any tasks 154 within any such phases 152 within the procedure. Procedure type 156 can include any information on type of the medical procedure performed, such as a name or designation of a medical procedure (e.g., open heart surgery, appendectomy, cholecystectomy, knee arthroscopy or a coronary artery bypass graft). Phases 152 can include any general portions of a medical procedure having multiple tasks 154. Phase 152 can include, for example, a pre-operative phase involving preparations for a surgery, an incision phase in which incisions are made to access an area on which to perform an action or an intervention, a surgical intervention phase in which medical interventions are implemented, a closure phase (e.g., suturing and hemostasis tasks) and a post-operative phase. Tasks 154 can include any events or actions taken to complete various phases 152 of the procedure. Tasks 154 can include preparation of surgical instruments, making of incisions, placing retractors or other instruments to maintain clear access, tissue manipulation or excision, administering anesthetics, wound suturing or other tasks within a medical procedure type 156.

Timestamp 162 can include any type and form of an indication of a time for marking a particular point, such as a starting time or ending time of a video data 136 for section or a portion of a medical procedure (e.g., medical procedure type 156). Timestamps 162 can include any combination of a millisecond, second, minute, hour, day, month or a year. Timestamps 162 can mark starting point, mid-point, ending point or any other temporal point of a task 154, phase 152, medical procedure start or end, starting or ending point of a description 166 or a label 164 or any other feature or object.

Labels 164 can include markings or labels for any portion of a video data 136. Labels 164 can include markings at particular points in time (e.g., at designated timestamps 162) for marking a particular task 154, phase 152 or a medical procedure. Labels 164 can mark phase 152, task 154 or procedure type 156, name, starting and ending points, titles, descriptions or any other designations or information. Labels 164 can be used to categorize different medical tasks or phases, indicating that a particular task is, for example, an incision, tissue manipulation or suturing. Labels 164 can mark various medical instruments 112 or features or objects identified in the video data 136.

Descriptions 166 can include any type and form of text describing any aspect of the medical procedure, including a procedure type 156, a phase 152 (e.g., phase type) or a task 154 (e.g., task type) or any indication, such as an indication of time (e.g., a timestamp 162), state 146, type of label 164 or any other data. Description 166 can include a textual description of a task 154, that can identify the type of a task, type of instruments 112 used, duration of the task, metrics 148 corresponding to the performance of the surgeon or any other relevant data or information. Description 166 can include annotations or commentary on what the surgeon is doing at particular tasks 154 or phases 152, providing information that can be overlaid over the video data 136 during the display.

Annotation interface function 180, also referred to as AIF 180, can include any combination of hardware and software for providing annotation interface 188 and its functionality. Annotation interface function 180 can include one or more expert review protocols 182 for providing review functionalities to various annotators (e.g., users) on devices 190. Annotation interface function 180 can include or utilize annotator accounts 184 to authenticate and authorize users (e.g., annotators) to access the annotation interface function 180 and the DPS 130. AIF 180 can include one or more levels of authentication and authorization, including user managed access (UMA) authorization, firewall functionality and a web-based authentication framework. Annotation interface function 180 can, for example, use username and password combinations to provide access to users, based on their respective annotator accounts 184. Annotator interface function 180 can provide access to annotation interface 188, including access to entries 142, annotation cards 160 and any procedure data 150 that any particular user associated with a given annotator account 184 may have access to view or edit. Annotator interface function 180 can provide functionality to users to access one or more ML models 172 to implement or facilitate any annotation related tasks, including any access to task and phase detectors 174, temporal functions 176 and metrics functions 178.

Annotation interface function 180 can provide any number of operation modes 402 for operating the annotation interface 188. Operation modes 402 can include a training mode of operation in which users or annotators can train or exercise their annotation skills. Training mode can include a training environment in which training videos can be provided. Training mode can allow annotators and surgeons to review video streams for education or training purposes. Operation modes 402 can include annotation mode in which the user can annotate various video streams either manually or using one or more ML models 172. AIF 180 can allow the users to toggle between the annotation modes and training modes of the annotation interface 188. AIF 180 can include the functionality, such as a gatekeeper, for assigning annotation duties to users based on their workload so as to load balance the work across various users. For example, the gatekeeper functionality of the AIF 180 can assign annotation duties based on user histories, including annotator data on prior annotation procedures, procedure types or other tasks performed by the user associated with the annotator account 184. The gatekeeper functionality of the AIF 180 can assign the annotation duties-based preference to provide particular users with more of a particular type of medical procedures, tasks 154 or phases 152 to annotate.

AIF 180 can include various functionalities to facilitate users with implementing their annotation duties. AIF 180 can include menu controls 306 to provide buttons, command prompts, or selections for users to select particular selections (e.g., tasks 154, phases 152, medical procedures, control the video player or assign timestamps 162). AIF 180 can include at tool bar 310 that can include a visualization tool to provide boundaries of particular phases 152, tasks 154 or portions of the procedure. For example, a tool bar 310 can provide rectangular strips along the width of the video file that can indicate or visualize time portions during which a particular task 154, phase 152 has occurred, or during which a particular medical instrument 112 has been used.

Expert review protocol 182 can include type and form of a protocol, procedure or arrangement for entering, filling, reviewing, validating or deleting any entries 142 associated with any annotation cards 160. Expert review protocol 182 can include identifiers for particular annotator accounts 184 that can access particular annotation cards 160 to validate or verify any particular entries 142 corresponding to a field 144 or state 146 for any particular timestamp 162, label 164 or description 166 of a phase 152, task 154 or a procedure (e.g., procedure type 156) as a whole. Expert review protocol 182 can include functionality to automatically distribute messages or requests to users associated with particular annotator accounts 184 to access given annotation cards 160. For example, expert review protocol 182 can determine, using annotator data 186, that a particular user associated with a given annotator account 184 has a history or expertise in dealing with particular types of medical procedures. Based on this determination, expert review protocol 182 can send a message to this particular annotator account 184 to ask the associated user to provide feedback (e.g., validation, review or analysis) of a particular annotation card 160, procedure data or entry 142.

Annotator accounts 184 can include any type and form of an account associated with a user, such as an annotator providing annotations to medical procedures. Annotator account 184 can include a profile of a user along with annotator data 186. Annotator data 186 can include the history or data about the user associated with the annotator account 184, including a history of the medical procedures that were annotated by the given user of the annotator account 184, the types of such procedures, the types of the associated tasks 154 or phases 152 and the familiarity of the user with given types of medical procedures. Annotator accounts 184 can receive access to various annotation cards 160 using or via authentication and authorization mechanisms, such as UMA authorization, web authentication framework to overcome any firewall functionalities.

The data processing system 130 can interface with, communicate with, or otherwise receive or provide information with one or more component of system 100 via network 101, including, for example, the RMS 120. The data processing system 130, RMS 120 and devices in the medical environment 102 can each include at least one logic device such as a computing device having a processor to communicate via the network 101. The DPS 130, any portion of the ML framework 170, the RMS 120 or a client device that can be communicatively coupled with the DPS or the RMS 120 via the network 101, can each include at least one computation resource, server, processor or memory for processing data. For example, the data processing system 130 can include a plurality of computation resources or processors coupled with memory.

The data processing system 130, as well as any of its components (e.g., ML framework 170, AIF 180) can each be a part of or include a cloud computing environment functionality or features. The data processing system 130 can include multiple, logically grouped servers and facilitate distributed computing techniques. The logical group of servers may be referred to as a data center, server farm or a machine farm. The servers can also be geographically dispersed. A data center or machine farm may be administered as a single entity, or the machine farm can include a plurality of machine farms. The servers within each machine farm can be heterogeneous—one or more of the servers or machines can operate according to one or more type of operating system platform.

The data processing system 130, or components thereof can include a physical or virtual computer system operatively coupled, or associated with, the medical environment 102. In some embodiments, the data processing system 130, or components thereof can be coupled, or associated with, the medical environment 102 via a network 101, either directly or directly through an intermediate computing device or system. The network 101 can be any type or form of network. The geographical scope of the network can vary widely and can include a body area network (BAN), a personal area network (PAN), a local-area network (LAN) (e.g., Intranet), a metropolitan area network (MAN), a wide area network (WAN), or the Internet. The topology of the network 101 can assume any form such as point-to-point, bus, star, ring, mesh, tree, etc. The network 101 can utilize different techniques and layers or stacks of protocols, including, for example, the Ethernet protocol, the internet protocol suite (TCP/IP), the ATM (Asynchronous Transfer Mode) technique, the SONET (Synchronous Optical Networking) protocol, the SDH (Synchronous Digital Hierarchy) protocol, etc. The TCP/IP internet protocol suite can include application layer, transport layer, internet layer (including, e.g., IPv6), or the link layer. The network 101 can be a type of a broadcast network, a telecommunications network, a data communication network, a computer network, a Bluetooth network, or other types of wired and wireless networks.

The data processing system 130, or components thereof, can be located at least partially at the location of the surgical facility associated with the medical environment 102 or remotely therefrom. Elements of the data processing system 130, or components thereof can be accessible via portable devices such as laptops, mobile devices, wearable smart devices, etc. The data processing system 130, or components thereof, can include other or additional elements that can be considered desirable to have in performing the functions described herein. The data processing system 130, or components thereof, can include, or be associated with, one or more components or functionality of a computing including, for example, one or more processors coupled with memory that can store instructions, data or commands for implementing the functionalities of the DPS 130 discussed herein.

Device 190 can include any type and form of a computing device, such as a user's personal computer, a smartphone or a laptop, that a user (e.g., annotator) associated with an annotator account 184 can use to access annotation interface function 180 an its functionalities. Device 190 can execute or operate an application 192 that can include any combination of hardware and software that can execute or utilize annotation interface function 180 and its functionalities from the device 190. Application 192 can include, for example, an agent or a remote access application that can execute or run an annotation interface 188 on the device 190, accessing the functionalities and services of the DPS 130. Application 192, can include, for example, a web browser application that can access an annotation interface 188 that can be implemented as a web-based application or interface to utilize annotation interface function 180 functionalities or features. Application 192 on a device 190 can run the annotation interface 188 to facilitate the access by a user associated with an annotator account 184 to various entries 142, procedure data 150, metrics 148 or any other functionalities or features of the DPS 130.

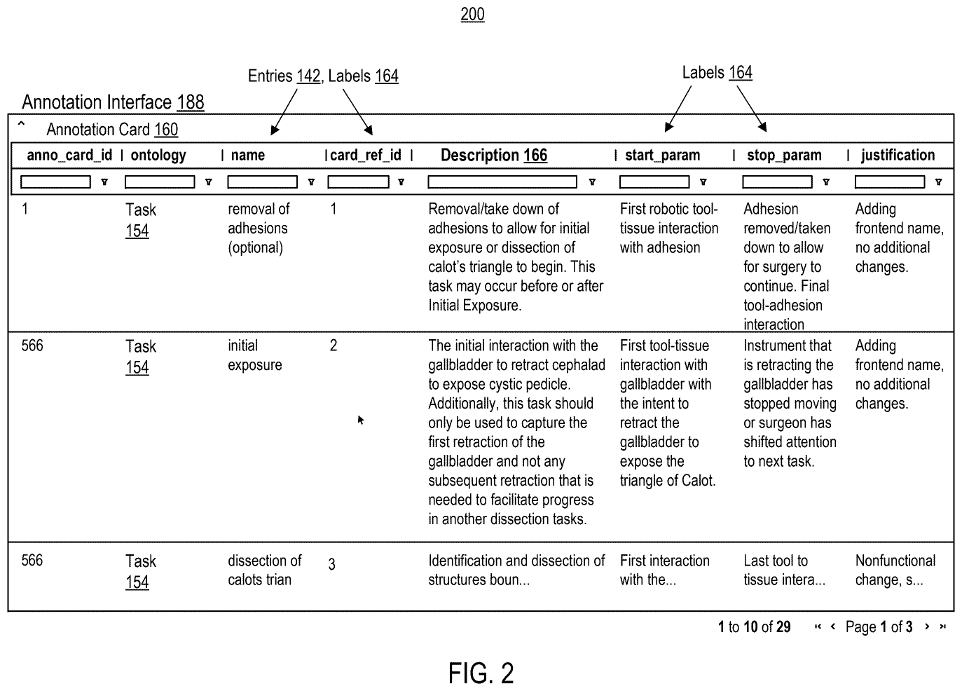

FIG. 2 illustrates an example 200 of one or more annotation cards 160 provided or displayed in an annotation interface 188. Annotation cards 160 can include any number of entries 142, such as labels 164 marking or labeling various metadata on tasks 154 or other portions of a procedure. Annotation cards 160 can include any procedure data 150, including any timestamps 162, labels 164 or descriptions 166. For instance, an annotation card 160 can include a description that includes a text describing actions taken during the course of a task 154 or a purpose of a particular task 154 in the course of a medical procedure. Annotation interface 188 can include any number of annotation cards 160 that can be identified using labels 164 corresponding to annotation card identifiers (e.g., IDs), ontology types (e.g., phases or tasks), card reference identifiers, start and stop parameters, comments (e.g., justifications) or any other information or procedure data 150.

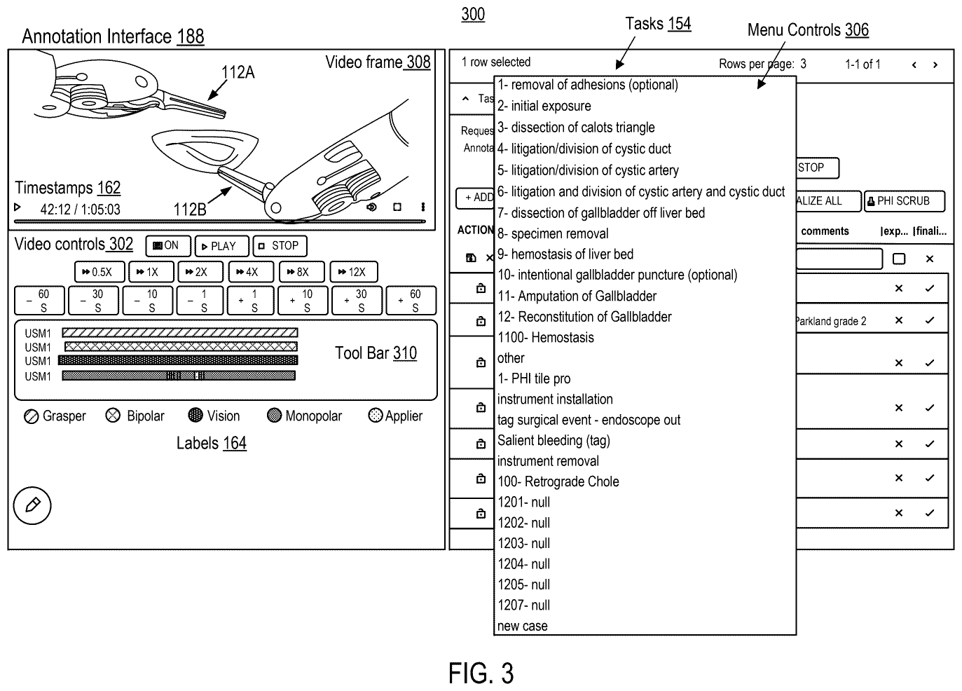

FIG. 3 illustrates an example 300 of an annotation interface 188 providing one or more video controls 302, user menus controls 306, video frames 308, tool bars 310 and labels 164 or timestamps 162 provided for selections of tasks 154 for user interaction in the annotation interface 188. Annotation interface 188 can include video controls 302 of a video streaming tool that can allow the users to watch the video stream being annotated. Video controls 302 of the video streaming tool displaying the video frames 308 can provide one or more menu controls 306 that can include selections, buttons or controls for the users (e.g., annotators) to press to select options (e.g., tasks 154 to assign to a label 164), or operate or control the video display (e.g., video frames 308) in the annotation interface 188. Video frames 308 can show medical instruments 112 (e.g., 112A and 112B) manipulating or interacting with an anatomy of a patient (e.g., a piece of a tissue or an organ). Video controls 302 can allow the user to scroll, fast forward, or reverse the video to any video frame 308 to apply a label 164 at any point in time (e.g., any timestamp 162) in the video stream (e.g., 136) in connection with any task 154 selected from a user menu 306.

Tool bar 310 can include visual indications of when a particular event in a medical procedure has occurred. Tool bar 310 can include a rectangular colored or patterned feature visually expressing the timing when an event took place. For example, tool bar 310 can show or indicate a timing when a particular medical tool (e.g., grasper) was being used during the procedure. Tool bar 310 can include several visual indicators to allow the user to visualize durations of particular tasks 154, phases 152, usage of medical instruments 112 or any other parameters, features or events during the medical procedure.

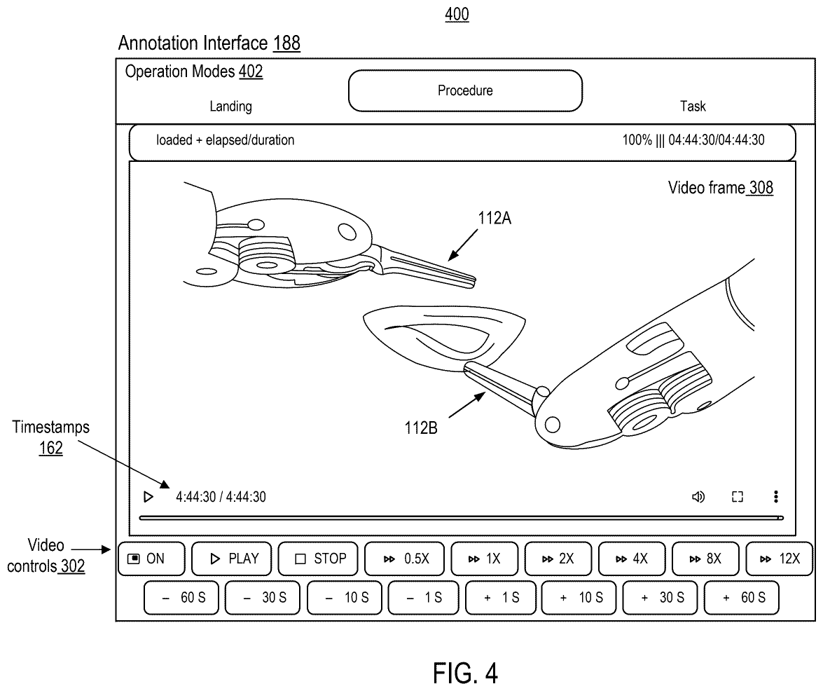

FIG. 4 illustrates an example 400 of an annotation interface 188 providing one or more operation modes 402 for the user to select to control, edit or manipulate video controls 302 or timestamps with respect to video frames 308 of a data stream. Operation mode 402 can include a procedure operation mode in which a user can create or edit entries 142, labels 164 or timestamps 162 for a procedure type 156, phase 152 or a task 154. Operation mode 402 can include a training mode in which a user can receive education or training on a particular task 154, phase 152 or procedure type 156 using the annotation interface 188 and its features. Annotation interface 188 can allow the user, operating in operation mode 402, to use video controls 302 to apply timestamps 162 and other procedure data 150 to create or edit entries 142 corresponding to any procedure data 150.

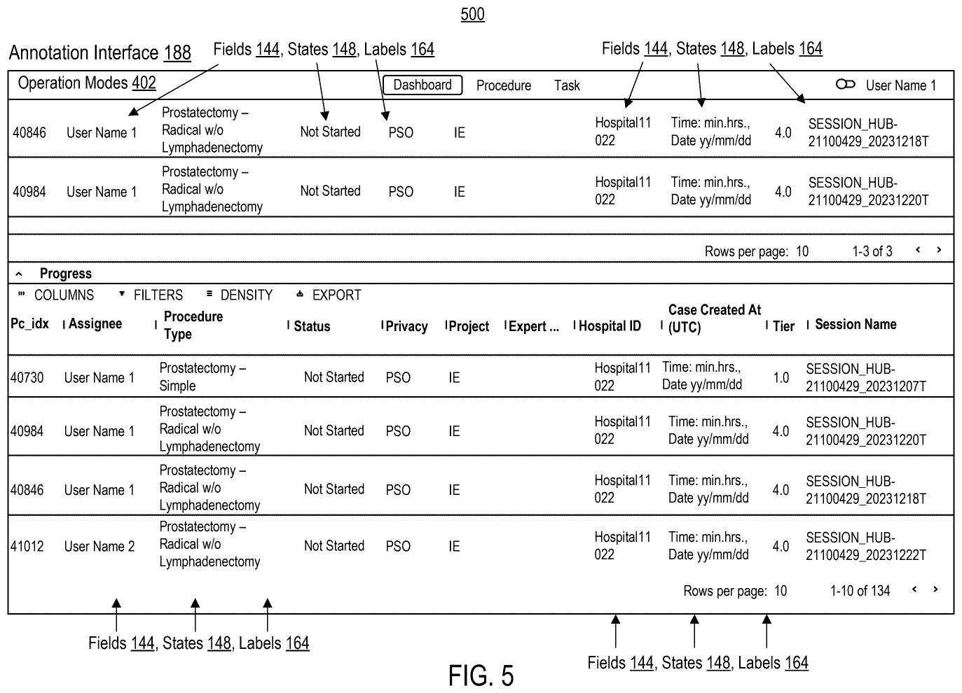

FIG. 5 illustrates an example 500 of an annotation interface 188 providing one or more operation modes 402 for the user to select to control, edit or manipulate fields 144 of entries 142, to enter, validate or modify states 146, labels 164 or other procedure data 150. Fields 144, states and labels 164 can be directed to, indicate or state a name or identifier of a hospital where a medical procedure took place, a time and date of the procedure, a session or video stream file name, a user name of an annotator providing labeling or annotation to the procedure, the state 148 of an entry 142 (e.g., not started) or any other information or data relevant to annotation.

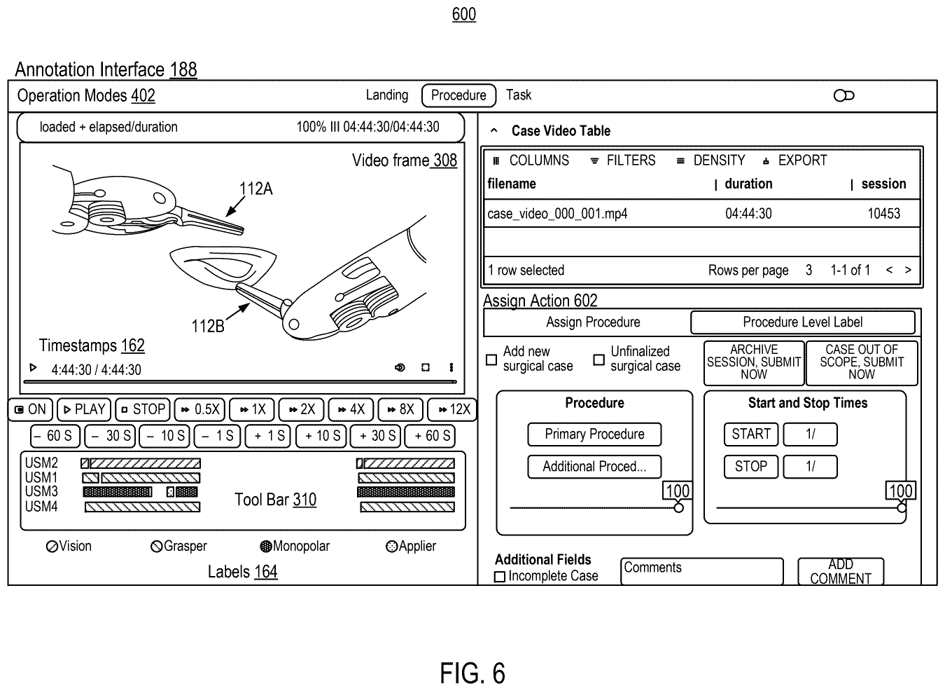

FIG. 6 illustrates an example 600 of an annotation interface 188 providing one or more operation modes 402 (e.g., procedure mode) to assign actions 602 to manipulate video frames 308 using video controls 302. Assign actions 602 can include an assignment of annotation of a procedure type 156, task 154 and phase 152 to a user associated with an annotator account. Assign action 602 can include adding a new surgical case, finalizing or reopening a case annotation, entering or editing start and stop times and adding comments. Assign actions 602 can be selected or implemented by the AIF 180 based on the workload or history of an annotator or a user to perform the annotation duties.

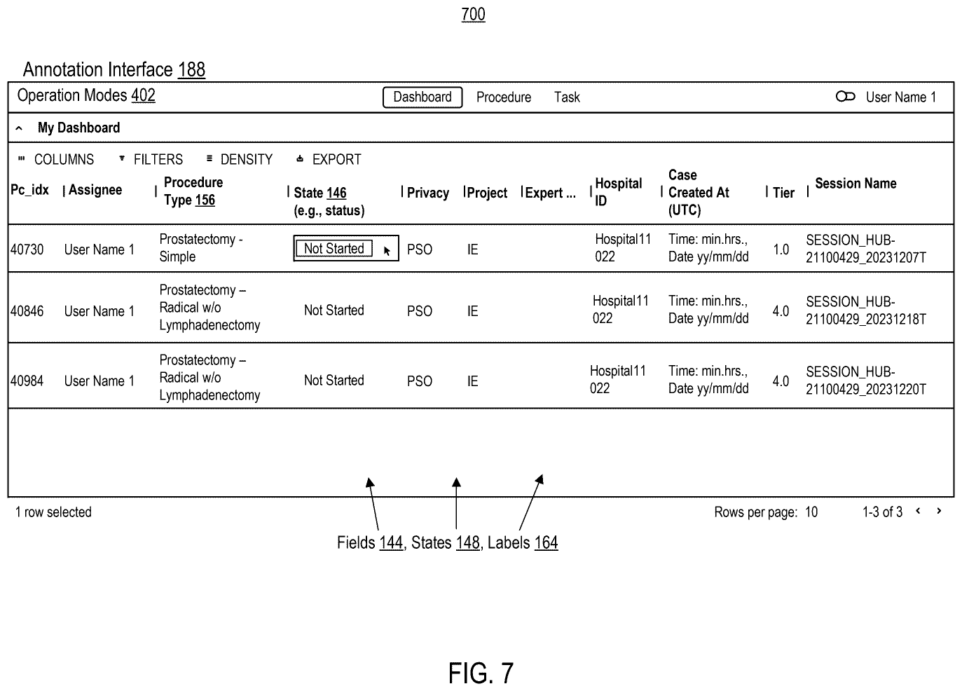

FIG. 7 illustrates an example 700 of an annotation interface 188 providing a dashboard view of one or more operation modes 402 to the user. In the dashboard view, the user can identify procedure types 156 along with their states 146 or status of the procedure in terms of annotation. The state 146 can indicate that a procedure type 156 (e.g., prostatectomy simple) was not yet started. Annotation interface 188 can provide fields 144, states 146 and labels 164 of various types, including hospital identifiers, time and date of the case created, tier (e.g., difficulty or complexity level) of the procedure, or session name of the procedure.

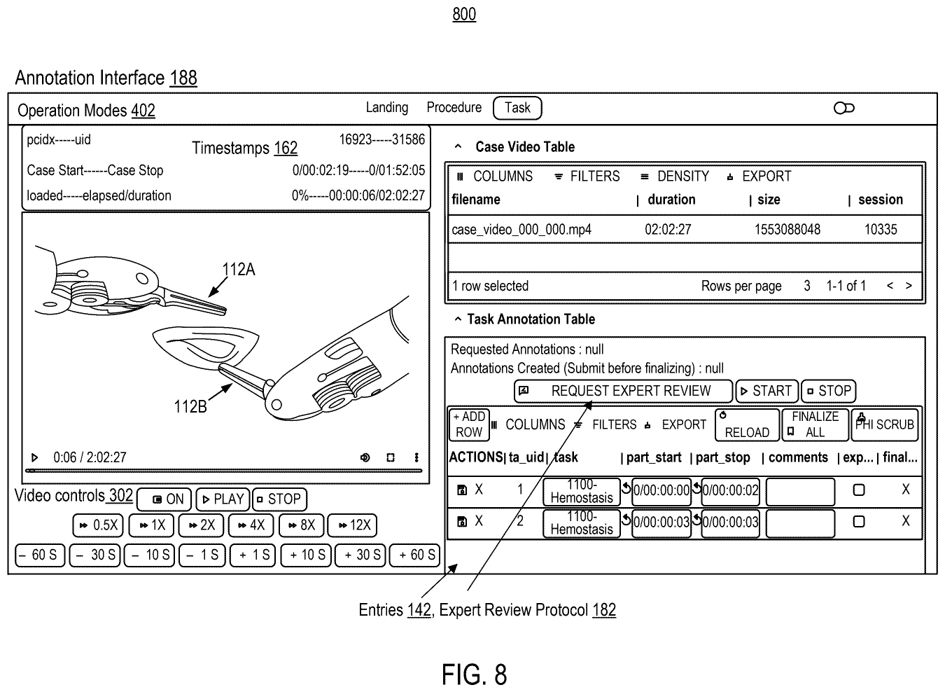

FIG. 8 illustrates an example 800 of an annotation interface 188 providing a task operation mode 402 in which a user (e.g., annotator) can select to control, edit or manipulate fields 144 of entries 142, to enter, validate or modify states 146, labels 164 or other procedure data 150 related to a task 154. Task related fields 144, states and labels 164 can be directed to, indicate or state a timestamp 162 (e.g., start and stop times), types of the task 154, comments about the task, or any other annotations about the task 154.

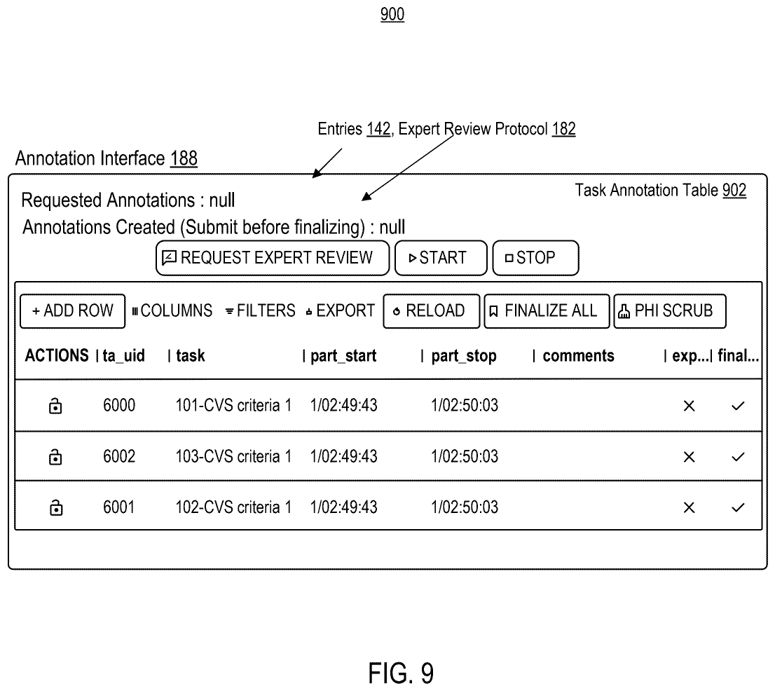

FIG. 9 illustrates an example 900 of an annotation interface 188 providing entries 142 for an expert review protocol 182 to assign users (e.g., expert level annotators for a particular procedure) to the annotation of the given procedure. Annotation interface 188 can provide a task annotation table 902 in which task 154, phases 152 or procedure types 156 for review can be listed. Task annotation table 902 can include assign actions 602 or information about users to perform the annotations. Entries 142 for expert level review can be sent using menu controls 306 (e.g., request expert review, start, stop, reload, add row or others). Expert review protocol 182 can involve assigning an annotation of the given portion of the procedure to a particular user or annotator whose annotation data 186 includes a history of annotations indicative that the user has had experience with a particular procedure type 156, phase 152 or a task 154. For instance, annotator data 186 can indicate that the user has had a number of similar or relevant annotation cases that is greater than a threshold (e.g., more than 15 annotation cases for a particular procedure type 156). Based on this determination, the ML model 172 or a user can assign the annotation task to the user associated with the given annotator account 184.

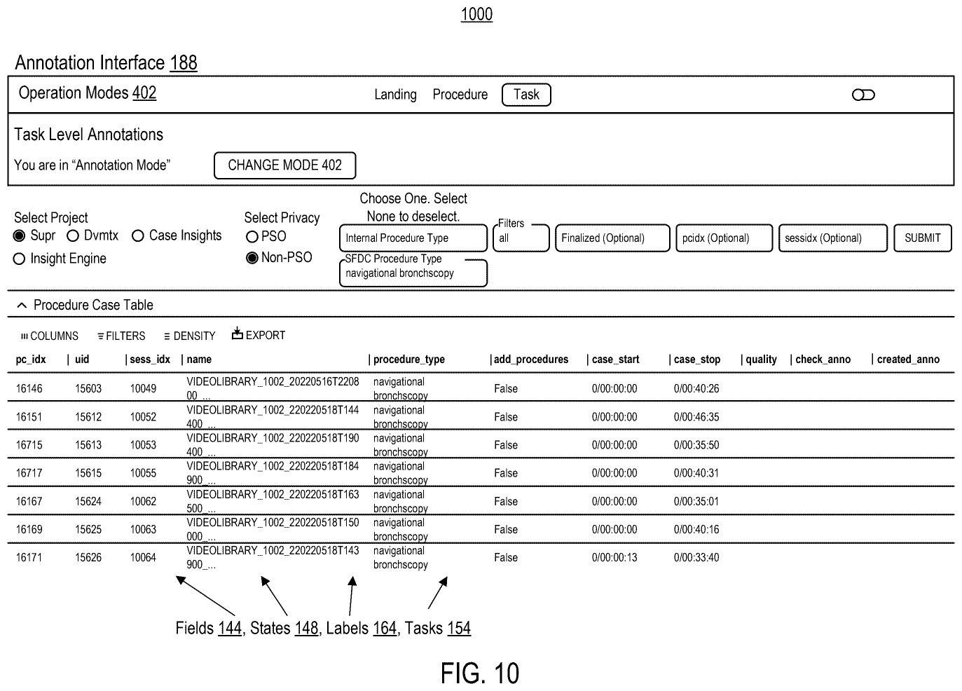

FIG. 10 illustrates an example 800 of an annotation interface 188 providing a task operation mode 402 in which a user (e.g., annotator) can select to control, edit or manipulate fields 144 of entries 142, to enter, validate or modify states 148, labels 164 or other procedure data 150 related to a task 154. Task related fields 144, states and labels 164 can be directed to, indicate or state a timestamp 162 (e.g., start and stop times), types of the task 154, comments about the task, or any other annotations about the task 154.

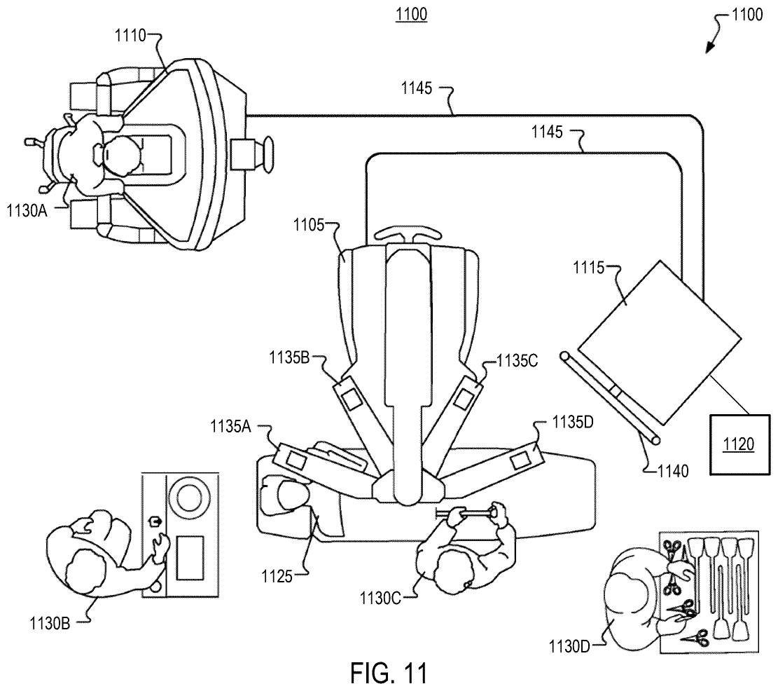

FIG. 11 depicts a surgical system 1100, in accordance with some embodiments. The surgical system 1100 may be an example of the medical environment 102. The surgical system 1100 may include a robotic medical system 1105 (e.g., the robotic medical system 120), a user control system 1110, and an auxiliary system 1115 communicatively coupled one to another. A visualization tool 1120 (e.g., the visualization tool 114) may be connected to the auxiliary system 1115, which in turn may be connected to the robotic medical system 1105. Thus, when the visualization tool 1120 is connected to the auxiliary system 1115 and this auxiliary system is connected to the robotic medical system 1105, the visualization tool may be considered connected to the robotic medical system. In some embodiments, the visualization tool 1120 may additionally or alternatively be directly connected to the robotic medical system 1105.

The surgical system 1100 may be used to perform a computer-assisted medical procedure on a patient 1125. In some embodiments, surgical team may include a surgeon 1130A and additional medical personnel 1130B-1130D such as a medical assistant, nurse, and anesthesiologist, and other suitable team members who may assist with the surgical procedure or medical session. The medical session may include the surgical procedure being performed on the patient 1125, as well as any pre-operative (e.g., which may include setup of the surgical system 1100, including preparation of the patient 1125 for the procedure), and post-operative (e.g., which may include clean up or post care of the patient), or other processes during the medical session. Although described in the context of a surgical procedure, the surgical system 1100 may be implemented in a non-surgical procedure, or other types of medical procedures or diagnostics that may benefit from the accuracy and convenience of the surgical system.

The robotic medical system 1105 can include a plurality of manipulator arms 1135A-1135D to which a plurality of medical tools (e.g., the medical tool 112) can be coupled or installed. Each medical tool can be any suitable surgical tool imaging device (e.g., an endoscope, an ultrasound tool, etc.), sensing instrument (e.g., a force-sensing surgical instrument), diagnostic instrument, or other suitable instrument that can be used for a computer-assisted surgical procedure on the patient 1125 (e.g., by being at least partially inserted into the patient and manipulated to perform a computer-assisted surgical procedure on the patient). Although the robotic medical system 1105 is shown as including four manipulator arms (e.g., the manipulator arms 1135A-1135D), in other embodiments, the robotic medical system can include greater than or fewer than four manipulator arms. Further, not all manipulator arms can have a medical tool installed thereto at all times of the medical session. Moreover, in some embodiments, a medical tool installed on a manipulator arm can be replaced with another medical tool as suitable.

One or more of the manipulator arms 1135A-1135D and/or the medical tools attached to manipulator arms can include one or more displacement transducers, orientational sensors, positional sensors, and/or other types of sensors and devices to measure parameters and/or generate kinematics information. One or more components of the surgical system 1100 can be configured to use the measured parameters and/or the kinematics information to track (e.g., determine poses of) and/or control the medical tools, as well as anything connected to the medical tools and/or the manipulator arms 1135A-1135D.

The user control system 1110 can be used by the surgeon 1130A to control (e.g., move) one or more of the manipulator arms 1135A-1135D and/or the medical tools connected to the manipulator arms. To facilitate control of the manipulator arms 1135A-1135D and track progression of the medical session, the user control system 1110 can include a display (e.g., the display 116 or 1130) that can provide the surgeon 1130A with imagery (e.g., high-definition 3D imagery) of a surgical site associated with the patient 1125 as captured by a medical tool (e.g., the medical tool 112, which can be an endoscope) installed to one of the manipulator arms 1135A-1135D. The user control system 1110 can include a stereo viewer having two or more displays where stereoscopic images of a surgical site associated with the patient 1125 and generated by a stereoscopic imaging system can be viewed by the surgeon 1130A. In some embodiments, the user control system 1110 can also receive images from the auxiliary system 1115 and the visualization tool 1120.

The surgeon 1130A can use the imagery displayed by the user control system 1110 to perform one or more procedures with one or more medical tools attached to the manipulator arms 1135A-1135D. To facilitate control of the manipulator arms 1135A-1135D and/or the medical tools installed thereto, the user control system 1110 can include a set of controls. These controls can be manipulated by the surgeon 1130A to control movement of the manipulator arms 1135A-1135D and/or the medical tools installed thereto. The controls can be configured to detect a wide variety of hand, wrist, and finger movements by the surgeon 1130A to allow the surgeon to intuitively perform a procedure on the patient 1125 using one or more medical tools installed to the manipulator arms 1135A-1135D.

The auxiliary system 1115 can include one or more computing devices configured to perform processing operations within the surgical system 1100. For example, the one or more computing devices can control and/or coordinate operations performed by various other components (e.g., the robotic medical system 1105, the user control system 1110) of the surgical system 1100. A computing device included in the user control system 1110 can transmit instructions to the robotic medical system 1105 by way of the one or more computing devices of the auxiliary system 1115. The auxiliary system 1115 can receive and process image data representative of imagery captured by one or more imaging devices (e.g., medical tools) attached to the robotic medical system 1105, as well as other data stream sources received from the visualization tool. For example, one or more image capture devices (e.g., the image capture devices 110) can be located within the surgical system 1100. These image capture devices can capture images from various viewpoints within the surgical system 1100. These images (e.g., video streams) can be transmitted to the visualization tool 1120, which can then passthrough those images to the auxiliary system 1115 as a single combined data stream. The auxiliary system 1115 can then transmit the single video stream (including any data stream received from the medical tool(s) of the robotic medical system 1105) to present on a display (e.g., the display 116) of the user control system 1110.

In some embodiments, the auxiliary system 1115 can be configured to present visual content (e.g., the single combined data stream) to other team members (e.g., the medical personnel 1130B-1130D) who might not have access to the user control system 1110. Thus, the auxiliary system 1115 can include a display 1140 configured to display one or more user interfaces, such as images of the surgical site, information associated with the patient 1125 and/or the surgical procedure, and/or any other visual content (e.g., the single combined data stream). In some embodiments, display 1140 can be a touchscreen display and/or include other features to allow the medical personnel 1130A-1130D to interact with the auxiliary system 1115.

The robotic medical system 1105, the user control system 1110, and the auxiliary system 1115 can be communicatively coupled one to another in any suitable manner. For example, in some embodiments, the robotic medical system 1105, the user control system 1110, and the auxiliary system 1115 can be communicatively coupled by way of control lines 1145, which can represent any wired or wireless communication link that can serve a particular implementation. Thus, the robotic medical system 1105, the user control system 1110, and the auxiliary system 1115 can each include one or more wired or wireless communication interfaces, such as one or more local area network interfaces, Wi-Fi network interfaces, cellular interfaces, etc. It is to be understood that the surgical system 1100 can include other or additional components or elements that can be needed or considered desirable to have for the medical session for which the surgical system is being used.

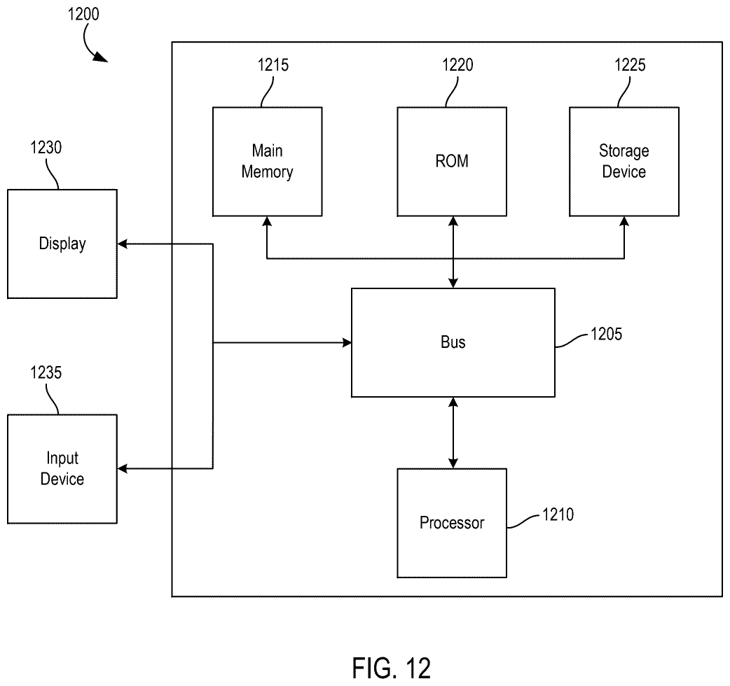

FIG. 12 depicts an example block diagram of an example computer system 1200 is shown, in accordance with some embodiments. The computer system 1200 can be any computing device used herein and can include or be used to implement a data processing system or its components. The computer system 1200 includes at least one bus 1205 or other communication component or interface for communicating information between various elements of the computer system. The computer system further includes at least one processor 1210 or processing circuit coupled to the bus 1205 for processing information. The computer system 1200 also includes at least one main memory 1215, such as a random-access memory (RAM) or other dynamic storage device, coupled to the bus 1205 for storing information, and instructions to be executed by the processor 1210. The main memory 1215 can be used for storing information during execution of instructions by the processor 1210. The computer system 1200 can further include at least one read only memory (ROM) 1220 or other static storage device coupled to the bus 1205 for storing static information and instructions for the processor 1210. A storage device 1225, such as a solid-state device, magnetic disk or optical disk, can be coupled to the bus 1205 to persistently store information and instructions.

The computer system 1200 can be coupled via the bus 1205 to a display 1230, such as a liquid crystal display, or active-matrix display, for displaying information. An input device 1235, such as a keyboard or voice interface can be coupled to the bus 1205 for communicating information and commands to the processor 1210. The input device 1235 can include a touch screen display (e.g., the display 1230). The input device 1235 can also include a cursor control, such as a mouse, a trackball, or cursor direction keys, for communicating direction information and command selections to the processor 1210 and for controlling cursor movement on the display 1230.

The processes, systems and methods described herein can be implemented by the computer system 1200 in response to the processor 1210 executing an arrangement of instructions contained in the main memory 1215. Such instructions can be read into the main memory 1215 from another computer-readable medium, such as the storage device 1225. Execution of the arrangement of instructions contained in the main memory 1215 causes the computer system 1200 to perform the illustrative processes described herein. One or more processors in a multi-processing arrangement can also be employed to execute the instructions contained in the main memory 1215. Hard-wired circuitry can be used in place of or in combination with software instructions together with the systems and methods described herein. Systems and methods described herein are not limited to any specific combination of hardware circuitry and software.

Although an example computing system has been described in FIG. 12, the subject matter including the operations described in this specification can be implemented in other types of digital electronic circuitry, or in computer software, firmware, or hardware, including the structures disclosed in this specification and their structural equivalents, or in combinations of one or more of them.

In one aspect, the technical solutions can include a system 100 that can include one or more processors (e.g., 1210) that can be coupled with memory (e.g., 1215 or 1220). The memory 1215 or 1220 can store instructions, computer code or data that can cause the one or more processors 1220 to implement any functionality of a DPS 130, including for example any functionality of a ML framework 170 and annotation interface function 180 to provide annotation of medical procedures. For instance, the memory 1215 or 1220 can store instructions, computer code or data to cause the one or more processors 1220 to provide users (e.g., annotators) associated with annotator accounts 184 with access and functionality to utilize, via annotation interface 188, any functionality of AIF 180 and ML framework 170, including ability to create, update, validate or provide feedback on any entries 142 for procedure data 150, such as phases 152, tasks 154 for any annotation card 160.

The system (e.g., 100) can include the one or more processors 1220 configured to receive at least a portion of a video stream 134 or 136 of a medical procedure performed during a medical session with a robotic medical system 120. The video data 136 stream can be stored in a repository 132 and accessed by a ML framework 170 or AIF 180. AIF 180 or ML framework 170 can access the video data 136 responsive to a selection, a request or an action by a user (e.g., annotator) associated with an annotator account 184. The one or more processors 1220 can access a plurality of video streams (e.g., 134) corresponding to a single medical procedure instance or a medical procedure type 156. The one or more processors 1220 can assemble or combine the plurality of video streams in user interface (e.g., annotation interface 188) to prepare the video stream of the medical procedure to run or display continuously from start to end of a medical procedure (e.g., 156) or across one or more phases 152 or one or more tasks 154. The combined video files may have no continuity between them in the storage and may be combined using AIF 180.

The system (e.g., 100) can include the one or more processors 1220 configured to identify, for the at least the portion of the video stream 134, a type of the medical procedure (e.g., 156) and a phase 152 of the medical procedure. The one or more processors 1220 can determine the procedure type 156 or the phase 152 based on a user selection or an input in an annotation interface 188. The one or more processors 1220 can determine the procedure type 156 or the phase 152 based on a determination or other actions by one or more ML models 172 that can be trained to determine, detect, recognize and annotate (e.g., timestamp, label or describe) procedure types 156, phases 152 or tasks 154. The ML model 172 can be trained to implement any annotation duties in real-time, including for example, labeling start and end temporal points of procedures, phases 152 or tasks 154 using timestamps 162, in real-time and during the course of the procedure. An ML trainer can train the ML model 172 based on the user selections, which the ML trainer can use to retrain the ML model 172 and improve the ML model's performance.

The system (e.g., 100) can include the one or more processors 1220 configured to determine, based on the type of the medical procedure (e.g., 156) and the phase 152 of the medical procedure, a plurality of types of tasks 154. The one or more processors 1220 can determine one or more type of tasks 154 based on a user selection or an input in an annotation interface 188. The one or more processors 1220 can determine the types of tasks 154 based on a determination or other actions by one or more ML models 172 that can be trained to determine, detect, recognize and annotate (e.g., timestamp, label or describe) one or more tasks 154 based on objects identified in video data 136, such as medical instruments 112 or patient anatomical parts or features.

The system (e.g., 100) can include the one or more processors 1220 configured to display an annotation interface 188 with the plurality of types of tasks 154. The annotation interface 188 can include a window or a feature for display of a video, as well as user selection tools (e.g., menu controls 306) for user to select and manipulate various procedure data 150, entries 142 or video stream (e.g., 136) features or portions. The system (e.g., 100) can include the one or more processors 1220 configured to receive, via the annotation interface, a selection of a first type of task 154 of the plurality of types of tasks 154 and an indication (e.g., timestamp 162) of a start time and a stop time for the first type of task 154. For example, a ML model 172 can be trained to analyze a portion of a video data 136 (e.g., video stream input into the ML model 172) and identify the timestamps 162 (e.g., temporal points) of the start time and end time of a phase 152, task 154 or a procedure type 156.

The system (e.g., 100) can include the one or more processors 1220 configured to identify frames (e.g., 308) of the at least the portion of the video stream (e.g., 136) that correspond to the start time and the stop time for the first type of task. For example, DPS 130 can select a video frame 308 in a video data 136 that corresponds to a timestamp 162 at which the particular task 154, phase 152 or procedure type 156 has started or ended. For example, DPS 130 can select a video frame 308 corresponding to the timestamps 162 using ML models 172 trained to identify the video frames 308 for the start or end of the phase 152, task 154 or procedure.

The system (e.g., 100) can include the one or more processors 1220 configured to construct, for storage in a data structure 140 for the medical session, an entry 142 that associates the frames (e.g., 308) that correspond to the start time and the stop time (e.g., timestamps 162) with an indication of the first type of task 154. The indication can include a label 164 indicative of the type or nature of the task 154. The label 164 can include a flag or a pointer to a start or end of the task 154 or a description 166 (e.g., textual explanation or a comment about the task 154).

The system (e.g., 100) can include the one or more processors 1220 configured to determine a state 146 of the entry 142 based on an expert review protocol 182 and update a field 144 in the entry 142 to indicate the state 146. For example, AIF 180 can determine that a state 146 of an entry 142 is at least one of incomplete, complete, assigned, unassigned, verified, unverified, awaiting annotation, in progress or any other. Expert review protocol 182 can be used to create an order of tasks by annotators or users with respect to the entries 142, including a first round of annotators assigning annotations of entries 142 and a second round of annotators validating or verifying the entries 142. The states 146 of the entries can be modified by the annotators (e.g., users) based on the expert review protocol 182.

The system (e.g., 100) can include the one or more processors 1220 configured to select an action to validate the entry 142 based on the state 146 and execute the action. The action can include an action or selection to make, analyze, validate an entry 142. The action can be selected to enter an annotation (e.g., timestamp 162, label 164 or description 166) for a field 144 of an entry 142. The state 146 of the entry can be changed upon completion of the entry of the annotation.

The system (e.g., 100) can include the one or more processors 1220 configured to forward, via a network 101, the entry 142 to a device 190 for validation. The device 190 can be a computer or a smartphone of a user (e.g., annotator) associated with an annotator account 184. The user can be a person assigned to the particular entry 142 based on an expert review protocol 182. The system (e.g., 100) can include the one or more processors 1220 configured to receive, via the network 101, a validation of the entry 142 according to a review by the user. The system (e.g., 100) can include the one or more processors 1220 configured to store, in the data structure 140, the entry 142 identified as validated.

The system 100 can include the one or more processors 1220 configured to forward, via a network 101, the entry 142 to a device 190 for validation. The system (e.g., 100) can include the one or more processors 1220 configured to receive, via the annotation interface 188 from the device 190, a modification to the entry 142. The entry modification can include a modification of any value or parameter in a field 144, including a timestamp 162, label 164, description 166 or any other metadata. The system (e.g., 100) can include the one or more processors 1220 configured to update the entry 142 based on the modification.

The system (e.g., 100) can include the one or more processors 1220 configured to identify a plurality of annotator accounts 184 associated with an expert review protocol 182. The system (e.g., 100) can include the one or more processors 1220 configured to select, based on annotation histories (e.g., annotation data 186) of the plurality of annotator accounts 184; a first account 184 of the plurality of accounts 184 to validate the entry 142. The annotator account 184 can be selected, for example, based on the annotator data 186 indicating that the user associated with the annotator account 184 has had more than a threshold number of cases corresponding to a particular task 154, phase 152, procedure type 156 or a group of related procedure types 156.

The system 100 can include the one or more processors 1220 configured to identify a plurality of previously validated entries 142 of a plurality of annotator accounts 184 associated with an expert review protocol 182. The system can include the one or more processors 1220 configured to determine, based on the plurality of previously validated entries 142 and the type of the medical procedure 156, a first annotator account 184 of the plurality of annotator accounts 184 to validate the entry 142.

The system 100 can include the one or more processors 1220 configured to identify a plurality of video stream files corresponding to the medical session. The system can include the one or more processors 1220 configured to combine the plurality of video stream files to form the at least the portion of the video stream (e.g., 136) of the medical procedure (150, 156). Multiple video files may include no continuity data between them and may include or correspond to different tasks 154 of different phases 152 of a medical procedure event or a medical procedure type 156. The system can include the one or more processors 1220 configured to display the at least portion of the video stream via the annotation interface 188.

The system 100 can include the one or more processors 1220 configured to identify, using the at least the portion of the video stream (e.g., 136), a plurality of phases 152 of the medical procedure comprising the phase 152. The system can include the one or more processors 1220 configured to identify, for each respective phase 152 of the plurality of phases 152, a start time (e.g., 162) of the each respective phase 152 and a stop time (e.g., 162) of the each respective phase 152. The system can include the one or more processors 1220 configured to construct, for storage in the data structure, a plurality of entries 142, each entry 142 of the plurality of entries 142 indicative of the start time (e.g., 162) of the each respective phase 152 and the stop time (e.g., 162) of the each respective phase 152.

The system 100 can include the one or more processors 1220 configured to provide, via the annotation interface 188, one or more operation modes 402 of the annotation interface 188. The system can include the one or more processors 1220 configured to display, responsive to a selection from the plurality of modes 402, a training mode 402 to provide training for annotation of the medical procedure. The operation mode 402 for training of the user can be used to train annotator users or train surgeons (e.g., medical professionals) on a particular task 154, phase 152 or procedure type 156.

The system 100 can include the one or more processors 1220 configured to provide, via the annotation interface 188, a plurality of annotation cards 160 for the plurality of types of tasks 154 of the phase 152 of the medical procedure. The system can include the one or more processors 1220 configured to display, via the annotation interface 188, responsive to a selection of a menu control 306 by a user associated with an annotator account 184, a first annotation card 160 of the plurality of annotation cards 160. The first annotation card 160 can be indicative of the start time (e.g., 162) and the stop time (e.g., 162) and can comprise a description 166 of the first type of task 154. The description 166 can provide explanation or information on the task 154 and its role in the procedure.

The system 100 can include the one or more processors 1220 configured to identify one or more machine learning (ML) models 172 trained on a plurality of video streams (e.g., 136) of a plurality of types of medical procedures having a plurality of phases 152 with a plurality of types of tasks 154. The system can include the one or more processors 1220 configured to identify at least one of the types of the medical procedure or the phase 152 of the medical procedure using the at least the portion of the video stream (e.g., 136) input into the one or more machine learning (ML) models 172.

The system 100 can include the one or more processors 1220 configured to identify one or more machine learning (ML) models 172 trained on a plurality of video streams (e.g., 136) of a plurality of types of medical procedures having a plurality of phases with a plurality of types of tasks identified by a plurality of start times and stop times. The system can include the one or more processors 1220 configured to identify the first type of task 154 and the indication of the start time and the stop time for the first type of task 154 using the one or more machine learning (ML) models 172.

The system 100 can include the one or more processors 1220 configured to identify one or more machine learning (ML) models 172 trained on a plurality of video streams (e.g., 136) of a plurality of types of medical procedures having a plurality of phases with a plurality of types of tasks. The system can include the one or more processors 1220 configured to determine, using the at least the portion of the video stream (e.g., 136) input into the one or more machine learning (ML) models 172, a metric 148 indicative of performance associated with a surgeon performing the medical procedure. The system can include the one or more processors 1220 configured to display the metric 148 via the annotation interface 188.

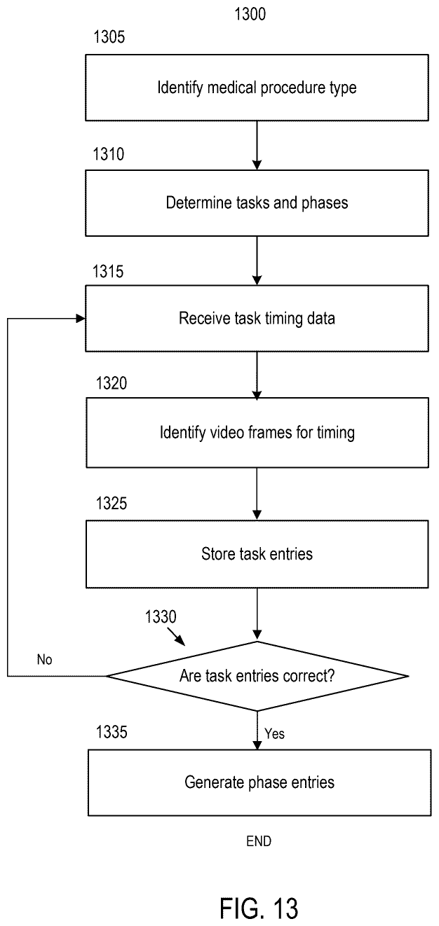

Turning now to FIG. 13, an example flow diagram of a method 1300 for annotation of medical procedures using a user interface and application framework is illustrated. The method 1300 can be performed by a system having one or more processors configured to perform operations of the system 100 by executing computer-readable instructions stored on a memory. The method can be implemented using a non-transitory computer readable medium storing instructions that, when executed by one or more processors, cause the one or more processors to implement operations of the method 1300. The method 1300 can be performed, for example, by system 100 and in accordance with any features or techniques discussed in connection with FIGS. 1-13. For instance, the method 1300 can be implemented one or more processors 1210 of a computing system 1200 executing non-transitory computer-readable instructions stored on a memory (e.g., the memory 1215, 1220 or 1225) and using data from a data repository 132 (e.g., storage device 1225).

The method 1300 can be used to provide medical procedure annotation using a video stream and a user interface. Method 1300 can include operations 1305-1335. At operation 1305, the method can identify medical procedure type. At operation 1310, the method can determine types of tasks and phases. At operation 1315, the method can receive task timing data. At operation 1320, the method can identify video frames for timing. At operation 1325, the method can store task entries. At operation 1330, the method can verify if the task entries are correct. At operation 1335, the method can generate phase entries.

At operation 1305, the method can identify medical procedure types. The medical procedure type can be received from a robotic medical system. The medical procedure type can be received responsive to a user request or responsive to a completion of the medical procedure at the robotic medical system. For instance, the method can identify the medical procedure and phase. The method can include one or more processors of a data processing system identifying a type of the medical procedure and a phase of the medical procedure based on one or more data streams generated by the robotic medical system during the course of performance of the medical procedure. The one or more processors can identify the type of the medical procedure, or the phase of the medical procedure based on, using or for at least a portion of a video stream of a medical procedure performed during a medical session with a robotic medical system. The one or more processors can determine the type of the medical procedure, or a type of the phase based on an entry by a user (e.g., annotator). The one or more processors can determine the type of the medical procedure, or the type of the phase based on a determination by one or more ML models.

The method can include the one or more processors identifying a plurality of video stream files corresponding to the medical session. The plurality of video files can correspond to a same task, phase or a medical procedure. The one or more processors can combine the plurality of video stream files to form the at least the portion of the video stream of the medical procedure a task or the phase. The method can include the one or more processors triggering, controlling or requesting displaying of the at least portion of the video stream via the annotation interface. The one or more processors can display the plurality of stream files as a single video file to present a video recording of a task, a phase or a medical procedure. The method can include the one or more processors identifying, using the at least the portion of the video stream, a plurality of phases of the medical procedure comprising the phase, or identifying one or more tasks of the phase or each of the plurality of phases.

At operation 1310, the method can determine one or more tasks and phases. The method can include the one or more processors utilizing one or more machine learning (ML) models to determine, predict one or more tasks or phases. The one or more determinations or predictions (e.g., annotations) of the tasks or phases can be stored in a database. The one or more processors can retrieve, from the database, the one or more ML-based determinations or predictions (e.g., annotations) of the one or more tasks or phases. The one or more processors can provide the retrieved determinations and predictions (e.g., annotations) of the one or more tasks or phases for display in an annotation interface.

The method can include a data processing system determining the types of tasks. For instance, the method can include the one or more processors determining, based on the type of the medical procedure and the phase of the medical procedure, a plurality of types of tasks. The plurality of tasks or the plurality of types of tasks can correspond to one or more phases of the medical procedure. The annotation interface function can utilize any combination of user inputs or menu selections along with ML modeling to determine the plurality of tasks or the plurality of types of tasks. For instance, the one or more processors can provide, via the annotation interface, a plurality of annotation cards for the plurality of types of tasks of the phase of the medical procedure. Users or annotators can select options or controls on the annotation card or the annotation interface to determine the types of tasks.

The method can include the one or more processors identifying one or more machine learning (ML) models trained on a plurality of video streams of a plurality of types of medical procedures having a plurality of phases with a plurality of types of tasks. The one or more ML models can be trained to detect types of tasks based on a determination of a phase of a medical procedure captured by the video stream. The one or more processors can identity, at least one of the types of the medical procedure or the phase of the medical procedure using the at least the portion of the video stream input into the one or more machine learning (ML) models. The one or more processors can identify, at least one of the type or phase of the medical procedure, based on entries of the users (e.g., annotators), including labels, timestamps or descriptions that can be input into the ML model to determine at least one of the phase or tasks of the identified phase.

At operation 1315, the method can receive task timing data. The method can include the one or more processors receiving, via an annotation interface displaying the plurality of types of tasks, a selection of a first type of task of the plurality of types of tasks and an indication of a start time and a stop time for the first type of task. The selection of the first type of the task can be determined based on a selection of a user utilizing the annotation interface. The indication of the start and stop time of the first type of task can be marked, labeled or detected based on a label that can include timestamps identifying timing in the video stream of the respective start and stop times.

The method can include the one or more processors identifying, for each respective phase of the plurality of phases, a start time of the each respective phase and a stop time of the each respective phase. The start and stop times can be indicated by labels or timestamps that can be entered in fields of entries of a data structure for an annotation card. For instance, each phase can include timestamps can be indicative of the start time and stop time. Start and stop times can indicate or mark start and stop temporal points of medical procedure, any one or more phases and any tasks within any of the phases. Entries can indicate medical instruments used in the procedures, phases or tasks. The one or more processors can identify one or more instruments and the indication for their timing or duration.

The method can include the one or more processors constructing, for storage in the data structure, a plurality of entries, each entry of the plurality of entries indicative of the start time of the each respective phase and the stop time of the each respective phase. The one or more processors can identify one or more machine learning (ML) models trained on a plurality of video streams of a plurality of types of medical procedures having a plurality of phases with a plurality of types of tasks identified by a plurality of start times and stop times. The method can include the one or more processors identifying the first type of task and the indication of the start time and the stop time for the first type of task using the one or more machine learning (ML) models.

The one or more processors can display, via the annotation interface responsive to a selection, a first annotation card of the plurality of annotation cards. The first annotation card can be indicative of the start time and the stop time and comprising a description of the first type of task. The first annotation card can identify the hospital location, user or annotator that has annotated or entered the entries of the annotation card, or any other information about timestamps, labels and descriptions utilized.

At operation 1320, the method can identify video frames for timing. The method can include the one or more processors identifying frames of the at least the portion of the video stream that correspond to the start time and the stop time for the first type of task. The video frames can correspond to the timestamps marking the starting temporal point and the ending temporal point of a medical procedure, medical phase or a medical task. The video frames can be identified based on matching of the timestamps data with the timing data of the video stream. The video frames can be identified based on inputs or selections of the users (e.g., annotators) utilizing the annotation interface 188, such as for example using entries in the fields of the data structure 140 (e.g., annotation card).

Video frames can be identified using, for example, one or more ML models that can be trained to identify the video frames based on various inputs. For example, video frames corresponding to start time and stop time can be determined based on the video stream data of the medical procedure or a task input into the ML model trained to identify the tasks from the video stream. For example, video frames corresponding to start time and stop time can be determined based on the entries of one or more data structures (e.g., from a series of chronologically ordered tasks) input into the ML model trained to identify each of the tasks from the video stream using the entries in the data structures. For example, a ML model can be trained to use one or more data structures corresponding to partially populated procedure data of a medical procedure and determine the tasks or phases across the medical procedure and its phases or tasks.

At operation 1325, the method can store one or more entries. The method can include storing, in a data structure for the medical session, an entry that associates the frames that correspond to the start time and the stop time with an indication of the first type of task. The method can include storing a plurality of entries for a plurality of tasks. The one or more entries can be stored along with their corresponding timestamps to indicate the temporal locations of the one or more tasks. The one or more processors can store the entries for the data structure in one or more databases of the storage. The one or more processors can store one or more entries for one or more tasks into a single data structure or can store entries of each task into a dedicated data structure for the task. The one or more processors can store one or more entries for one or more phases into a single data structure or can store entries of each phase (e.g., including its tasks) into a dedicated data structure for the given phase. The one or more processors can store one or more entries for one or more tasks into a single data structure for a medical procedure. The single data structure for the medical procedure can include databases or tables for various phases or tasks.

At operation 1330, the method can verify if the one or more entries is correct. For example, the one or more processors can provide the one or more predictions or determinations (e.g., annotations) of the one or more tasks or phases for display on an annotation interface. The one or more processors can receive one or more inputs with respect to the one or more predictions or determinations (e.g., annotations) via the annotation interface. The one or more processors can correct the one or more determinations or predictions (e.g., annotations) of the one or more tasks based on the one or more inputs. The one or more inputs can include, for example, and adjustment or a correction to start time or end time of a task (e.g., task annotation), an adjustment or a correction to a type of a task annotation, a name of the task, a description of a task, a comment for a task, or any other feature of an annotation card corresponding to the task.

The method can include the one or more processors determining if the one or more task entries stored in the data structure is correct, incorrect, valid, invalid, verified or unverified. The one or more processors can determine a state of the entry based on an expert review protocol and update a field in the entry to indicate the state. For instance, the expert review protocol can identify a particular user (e.g., an expert in the type of the procedure) to review the ML-based determinations or predictions of task annotations. The method can include the one or more processors selecting an action to validate the entry based on the state and executing the action. For instance, responsive to an input via an annotation interface, the one or more processors can determine that the ML-based prediction or determination is validated. The action can include any action of a menu control, any user selection or any input from a user or annotator. Responsive to user selections of the menu controls, the one or more processors can adjust ML-based predictions or determinations of the tasks and validate adjusted annotations per user selections or incorporating user input (e.g., descriptions, comments, corrections to start time and stop time for tasks or other annotations).

The method can include the one or more processors forwarding, via a network, the entry to a device for validation. The entry can be sent to a particular user or annotator based on a determination of the workload of all the available annotators for the given entry. For example, based on the expert review protocol, the annotation interface function can determine that a user has more availability than other users and the system can load balance (e.g., provide the entry to least busy annotator). The one or more processors can receive, via the network, a validation of the entry according to a review. For instance, the annotator can provide the validation and send it back to the DPS. The one or more processors can store, in the data structure, the entry identified as validated.

The method can include the one or more processors forwarding, via a network, the entry to a device for validation. The one or more processors can receive, via the annotation interface from the device, a modification to the entry. The one or more processors can update the entry based on the modification. The one or more processors can identify a plurality of accounts associated with an expert review protocol. The one or more processors can select, based on annotation histories of the plurality of accounts, a first account of the plurality of accounts to validate the entry. The annotation history can provide the current level of user's workload, the users prior annotated medical procedures, the number of medical procedures, tasks or phases of each type that the user has annotated, the level of familiarity with different medical procedures, phases or tasks. Based on this information, the annotation interface function can determine the annotator account to assign the particular annotation assignment.

For example, the method can include the one or more processors identifying a plurality of previously validated entries of a plurality of accounts associated with an expert review protocol. The one or more processors can determine, based on the plurality of previously validated entries and the type of the medical procedure, a first account of the plurality of accounts to validate the entry. For instance, the one or more processors can provide, via the annotation interface, a plurality of modes of the annotation interface. The one or more processors can display, by the one or more processors, responsive to a selection from the plurality of modes, a training mode to provide training for annotation of the medical procedure. The one or more processors can identify one or more machine learning (ML) models trained on a plurality of video streams of a plurality of types of medical procedures having a plurality of phases with a plurality of types of tasks. The one or more processors can determine using the at least the portion of the video stream input into the one or more machine learning (ML) models, a metric indicative of performance associated with a surgeon performing the medical procedure. The metric can be a metric of performance of the surgeon or a medical professional with respect to a particular task, phase or a medical procedure. The one or more processors can display the metric of performance via the annotation interface.

At the end of act 1330, the method can determine or verify if the one or more entries are correct or if the one or more entries are to be updated. For example, the one or more processors can determine if all of the entries for all of the tasks processed by the one or more ML models are reviewed or verified by one or more users (e.g., annotators) per expert review protocol. To the extent that the one or more processors determine that not all of the tasks processed by the one or more ML models are yet processed or verified, or that one or more task entries are incorrect or yet to be verified, then the process can go back to task 1315 to redo the tasks 1315-1330. To the extent that the one or more processors determine that all of the tasks processed by the one or more ML models are also reviewed and verified by one or more users, per expert review protocol, the method can move on to task 1335.

At operation 1335, the method can generate phase entries. For example, the method can include generating, by the one or more processors, one or more entries that include one or more annotations for a phase of the medical procedure. For example, the one or more processors can automatically determine the one or more entries with one or more annotations for one or more phases of the tasks that are determined to be verified or correct at operation 1330. The one or more entries of the one or more phases can be determined based on, using, or otherwise according to the verified entries of the one or more tasks.

The one or more annotations for a phases can include start time or end time of the phase, a name of the phase, a commentary or a description of the phase or any other information about the phase. The one or more processors can provide the one or more phase entries for display in the annotation interface. The displayed one or more phase entries can provide illustration, indication or a graphic presenting the phase along with any annotations about the phase or any annotations for any of the tasks within the phase.

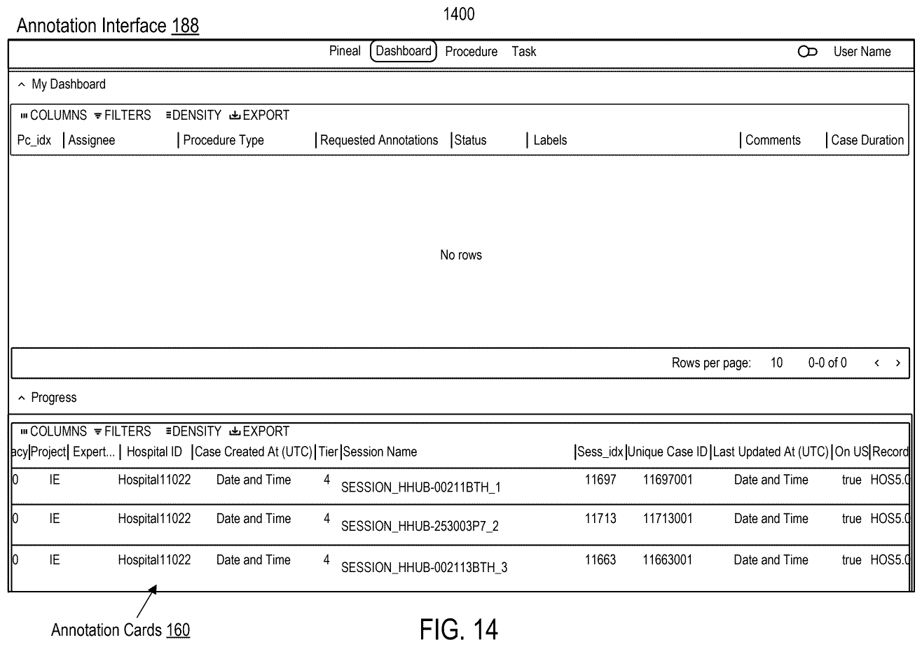

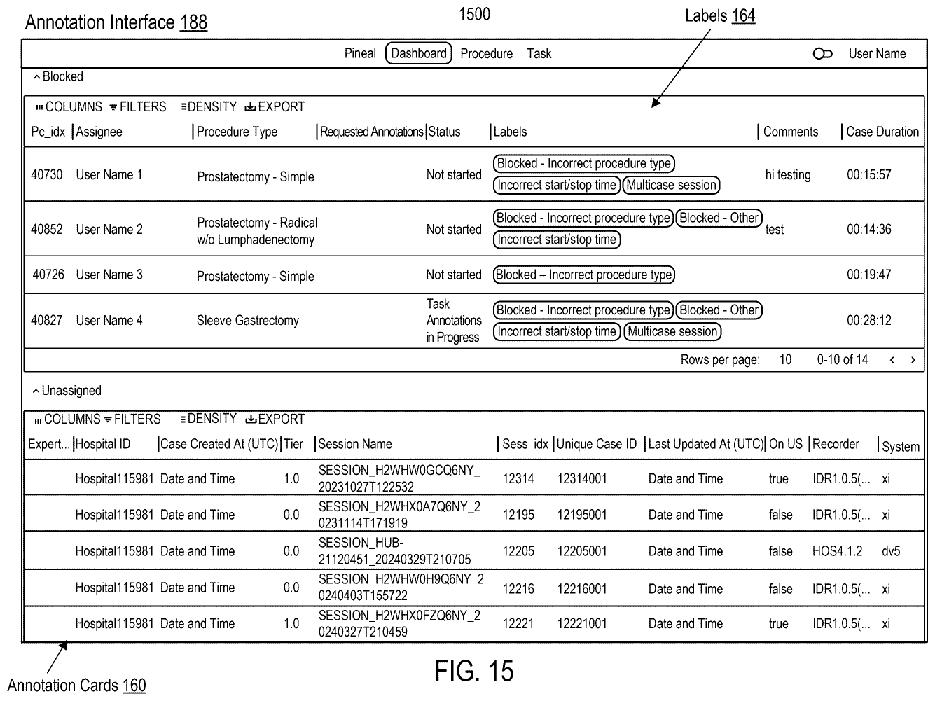

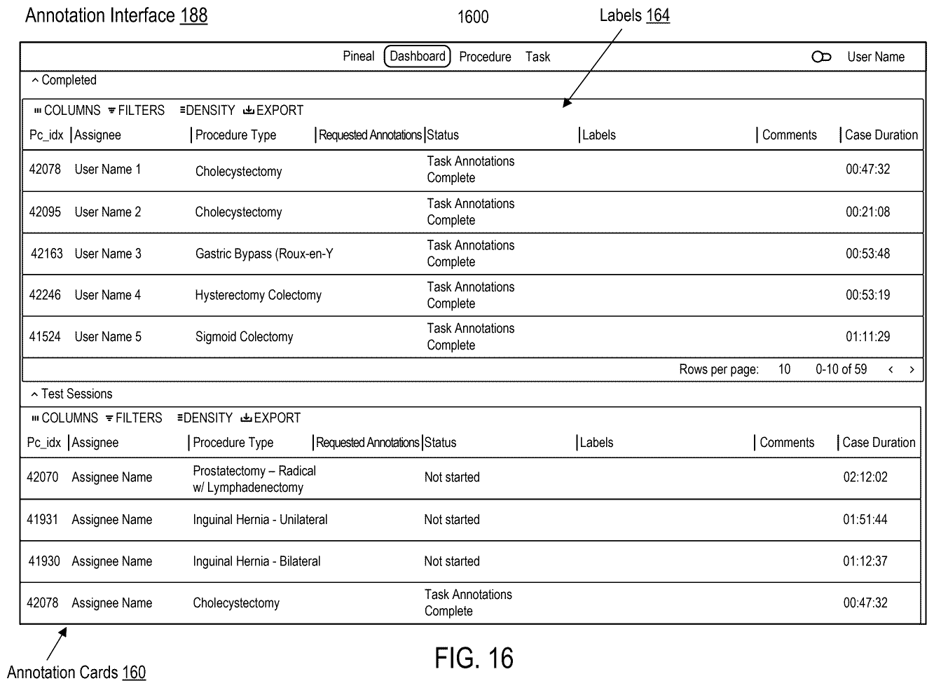

FIGS. 14-16 illustrate examples 1400-1600 of one or more annotation interfaces 188 that can allow users to interact with various annotation cards 160 and their components (e.g., labels 164). For example, FIG. 14 illustrates an example 1400 of an annotation interface 188 showing a dashboard functionality and features in which the user can interact with various annotation cards 160. For example, FIG. 15 illustrates an example 1500 of an annotation interface 188 in which a user can select various annotation cards 160 and interact with different example labels 164. For example, FIG. 16 illustrates an example 1600 of an annotation interface 188 in which the user can select different sets of annotation cards 160 and interact with different types of labels 164 or entries.

The herein described subject matter sometimes illustrates different components contained within, or connected with, different other components. It is to be understood that such depicted architectures are illustrative, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable or physically interacting components or wirelessly interactable or wirelessly interacting components or logically interacting or logically interactable components.

With respect to the use of plural or singular terms herein, those having skill in the art can translate from the plural to the singular or from the singular to the plural as is appropriate to the context or application. The various singular/plural permutations can be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.).

Although the figures and description can illustrate a specific order of method steps, the order of such steps can differ from what is depicted and described, unless specified differently above. Also, two or more steps can be performed concurrently or with partial concurrence, unless specified differently above. Such variation can depend, for example, on the software and hardware systems chosen and on designer choice. All such variations are within the scope of the disclosure. Likewise, software implementations of the described methods can be accomplished with standard programming techniques with rule-based logic and other logic to accomplish the various connection steps, processing steps, comparison steps, and decision steps.

It will be further understood by those within the art that if a specific number of an introduced claim recitation is intended, such an intent will be explicitly recited in the claim, and in the absence of such recitation, no such intent is present. For example, as an aid to understanding, the following appended claims can contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, or A, B, and C together, etc.). In those instances where a convention analogous to “at least one of A, B, or C, etc.” is used, in general, such a construction is intended in the sense one having skill in the art would understand the convention (e.g., “a system having at least one of A, B, or C” would include but not be limited to systems that have A alone, B alone, C alone, A and B together, A and C together, B and C together, or A, B, and C together, etc.). It will be further understood by those within the art that virtually any disjunctive word or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Further, unless otherwise noted, the use of the words “approximate,” “about,” “around,” “substantially,” etc., mean plus or minus ten percent.

The foregoing description of illustrative implementations has been presented for purposes of illustration and of description. It is not intended to be exhaustive or limiting with respect to the precise form disclosed, and modifications and variations are possible in light of the above teachings or can be acquired from practice of the disclosed implementations. It is intended that the scope of the invention be defined by the claims appended hereto and their equivalents.

What is claimed is:

1. A system comprising:
one or more processors, coupled with memory, to:
receive at least a portion of a video stream of a medical procedure performed during a medical session with a robotic medical system;
identify, for the at least the portion of the video stream, a type of the medical procedure and a phase of the medical procedure;
determine, based on the type of the medical procedure and the phase of the medical procedure, a plurality of types of tasks;
display an annotation interface with the plurality of types of tasks;
receive, via the annotation interface, a selection of a first type of task of the plurality of types of tasks and an indication of a start time and a stop time for the first type of task;
identify frames of the at least the portion of the video stream that correspond to the start time and the stop time for the first type of task; and
construct, for storage in a data structure for the medical session, an entry that associates the frames that correspond to the start time and the stop time with an indication of the first type of task.

2. The system of claim 1, comprising the one or more processors to:
determine a state of the entry based on an expert review protocol; and
update a field in the entry to indicate the state.

3. The system of claim 2, comprising the one or more processors to:
select an action to validate the entry based on the state; and
execute the action.

4. The system of claim 1, comprising the one or more processors to:
forward, via a network, the entry to a device for validation;
receive, via the network, a validation of the entry according to a review; and
store, in the data structure, the entry identified as validated.

5. The system of claim 1, comprising the one or more processors to:
forward, via a network, the entry to a device for validation;
receive, via the annotation interface from the device, a modification to the entry; and
update the entry based on the modification.

6. The system of claim 1, comprising the one or more processors to:
identify a plurality of accounts associated with an expert review protocol; and
select, based on annotation histories of the plurality of accounts; a first account of the plurality of accounts to validate the entry.

7. The system of claim 1, comprising the one or more processors to:
identify a plurality of previously validated entries of a plurality of accounts associated with an expert review protocol; and
determine, based on the plurality of previously validated entries and the type of the medical procedure, a first account of the plurality of accounts to validate the entry.

8. The system of claim 1, comprising the one or more processors to:
identify a plurality of video stream files corresponding to the medical session;
combine the plurality of video stream files to form the at least the portion of the video stream of the medical procedure; and
display the at least portion of the video stream via the annotation interface.

9. The system of claim 1, comprising the one or more processors to:
identify, using the at least the portion of the video stream, a plurality of phases of the medical procedure comprising the phase;
identify, for each respective phase of the plurality of phases, a start time of the each respective phase and a stop time of the each respective phase; and
construct, for storage in the data structure, a plurality of entries, each entry of the plurality of entries indicative of the start time of the each respective phase and the stop time of the each respective phase.

10. The system of claim 1, comprising the one or more processors to:
provide, via the annotation interface, a plurality of modes of the annotation interface; and
display, responsive to a selection from the plurality of modes, a training mode to provide training for annotation of the medical procedure.

11. The system of claim 1, comprising the one or more processors to:
provide, via the annotation interface, a plurality of annotation cards for the plurality of types of tasks of the phase of the medical procedure; and
display, via the annotation interface, responsive to a selection, a first annotation card of the plurality of annotation cards, the first annotation card indicative of the start time and the stop time and comprising a description of the first type of task.

12. The system of claim 1, comprising the one or more processors to:
identify one or more machine learning (ML) models trained on a plurality of video streams of a plurality of types of medical procedures having a plurality of phases with a plurality of types of tasks; and
identify at least one of the type of the medical procedure or the phase of the medical procedure using the at least the portion of the video stream input into the one or more machine learning (ML) models.

13. The system of claim 1, comprising the one or more processors to:
identify one or more machine learning (ML) models trained on a plurality of video streams of a plurality of types of medical procedures having a plurality of phases with a plurality of types of tasks identified by a plurality of start times and stop times; and
identify the first type of task and the indication of the start time and the stop time for the first type of task using the one or more machine learning (ML) models.

14. The system of claim 1, comprising the one or more processors to:
identify one or more machine learning (ML) models trained on a plurality of video streams of a plurality of types of medical procedures having a plurality of phases with a plurality of types of tasks;
determine, using the at least the portion of the video stream input into the one or more machine learning (ML) models, a metric indicative of performance associated with a surgeon performing the medical procedure; and
display the metric via the annotation interface.

15. A method, comprising
identifying, by one or more processors, for at least a portion of a video stream of a medical procedure performed during a medical session with a robotic medical system, a type of the medical procedure and a phase of the medical procedure;
determining, by the one or more processors, based on the type of the medical procedure and the phase of the medical procedure, a plurality of types of tasks;
receiving, by the one or more processors, via an annotation interface displaying the plurality of types of tasks, a selection of a first type of task of the plurality of types of tasks and an indication of a start time and a stop time for the first type of task;
identifying, by the one or more processors, frames of the at least the portion of the video stream that correspond to the start time and the stop time for the first type of task; and
storing, in a data structure for the medical session, an entry that associates the frames that correspond to the start time and the stop time with an indication of the first type of task.

16. The method of claim 15, comprising:
determining, by the one or more processors, a state of the entry based on an expert review protocol; and
updating, by the one or more processors, a field in the entry to indicate the state.

17. The method of claim 16, comprising:
selecting, by the one or more processors, an action to validate the entry based on the state; and
executing, by the one or more processors, the action.

18. The method of claim 15, comprising:
forwarding, by the one or more processors via a network, the entry to a device for validation;
receiving, by the one or more processors via the network, a validation of the entry according to a review; and
storing, by the one or more processors in the data structure, the entry identified as validated.

19. The method of claim 15, comprising:
forwarding, by the one or more processors via a network, the entry to a device for validation;
receiving, by the one or more processors via the annotation interface from the device, a modification to the entry; and
updating, by the one or more processors, the entry based on the modification.

20. A non-transitory computer-readable medium storing processor executable instructions that, when executed by one or more processors, cause the one or more processors to:
receive at least a portion of a video stream of a medical procedure performed during a medical session with a robotic medical system;
identify, for the at least the portion of the video stream, a type of the medical procedure and a phase of the medical procedure;
determine, based on the type of the medical procedure and the phase of the medical procedure, a plurality of types of tasks;
display an annotation interface with the plurality of types of tasks;
receive, via the annotation interface, a selection of a first type of task of the plurality of types of tasks and an indication of a start time and a stop time for the first type of task;
identify frames of the at least the portion of the video stream that correspond to the start time and the stop time for the first type of task; and
construct, for storage in a data structure for the medical session, an entry that associates the frames that correspond to the start time and the stop time with an indication of the first type of task.
